(19) United States
(12) Patent Application Publication
(10) Pub. No.: US 2023/0331652 A1
(43) Pub. Date: Oct. 19, 2023

(54) TRANS-CYCLOOCTENES WITH HIGH REACTIVITY AND FAVORABLE PHYSIOCHEMICAL PROPERTIES

(71) Applicants: Joseph M. Fox, Landenberg, PA (US); Jessica Pigga, Pawcatuck, CT (US); Samantha Jo Boyd, Wilmington, DE (US)

(72) Inventors: Joseph M. Fox, Landenberg, PA (US); Jessica Pigga, Pawcatuck, CT (US); Samantha Jo Boyd, Wilmington, DE (US)

(73) Assignee: University of Delaware, Newark, DE (US)

(21) Appl. No.: 18/210,862

(22) Filed: Jun. 16, 2023

Related U.S. Application Data
(63) Continuation of application No. PCT/US2021/064051, filed on Dec. 17, 2021.
(60) Provisional application No. 63/126,558, filed on Dec. 17, 2020.

(51) Int. Cl. C07C 49/607; C07C 35/20; C07C 35/21; C07D 311/82; C07D 207/408; C07C 29/143

(52) U.S. Cl. CPC C07C 49/607; C07C 35/20; C07C 35/21; C07D 311/82; C07D 207/408; C07C 29/143

(57) ABSTRACT

The present invention discloses a new class of trans-cyclooctenes (TCOs), “a-TCOs,” that are prepared in high yield via stereocontrolled 1,2-additions of nucleophiles to trans-cyclooct-4-enone, which itself was prepared on large scale in two steps from 1,5-cyclooctadiene. Computational transition state models rationalize the diastereoselectivity of 1,2-additions to deliver a-TCO products, which were also shown to be more reactive than standard TCOs and less hydrophobic than even a trans-oxocene analog. Illustrating the favorable physicochemical properties of a-TCOs, a fluorescent TAMRA derivative in live HeLa cells was shown to be cell-permeable through intracellular Diels-Alder chemistry and to washout more rapidly than other TCOs.

### CROSS-REFERENCE TO RELATED APPLICATIONS

This is a continuation application of International Application No. PCT/US2021/064051, filed on Dec. 17, 2021, which claims priority to U.S. Provisional Patent Application No. 63/126,558, filed on Dec. 17, 2020, the disclosures of each of these applications being incorporated herein by reference in their entireties for all purposes.

### STATEMENT REGARDING FEDERALLY SPONSORED RESEARCH

This invention was made with government support under Grant No. GM132460 awarded by the National Institutes of Health. The government has certain rights in the invention.

### BACKGROUND OF THE INVENTION

Bioorthogonal reactions are a class of rapid, selective reactions that can proceed efficiently and selectively in biological systems without interfering with biological functional groups. Bioorthogonal chemistry has enabled a deeper understanding of native biological processes and expanded the frontiers of chemical biology through innovations in nuclear medicine, drug delivery, and biomaterials. The tetrazine ligation with trans-cyclooctenes (TCOs) has been at the forefront of bioorthogonal methodologies due to rapid reaction kinetics that generally exceed k₂ 10⁴ M⁻¹s⁻¹.

TCO derivatives are synthesized by a photochemical flow-method under singlet sensitized conditions, driving an otherwise unfavorable isomeric ratio in favor of the trans-isomer via selective metal complexation to Ag(I) (M. Royzen, G. P. A. Yap, 3. M. Fox, J. Am. Chem. Soc. 2008, 130, 3760-3761). Flow photoisomerization have been developed where flow is mimicked by periodically stopping irradiation and capturing the TCO product by filtering through AgNO₃-silica and re-subjecting the filtrate to photoisomerization (at 254 nm). While this method has been used to synthesize a large number of different TCO derivatives, the production of diastereomers due to the planar chirality of the alkene presents a bottleneck for the majority of TCO syntheses. For example, the most frequently utilized TCO derivatives are the axial and equatorial diastereomers of 5-hydroxy-trans-cyclooctene, which are produced through photoisomerization of 5-hydroxy-cis-cyclooctene in 72% yield. Derivatives of the axial diastereomer have emerged as especially useful, as they are an order of magnitude more reactive than equatorial diastereomers, can promote fluorogenic effects for cell imaging, and have been employed in ‘click-to-release’ strategies for bioorthogonal uncaging. However, photoisomerization produces the equatorial:axial isomers in a 2.2:1 ratio, resulting in 24% of the axial diastereomer. A modified procedure using Ag(I) sulfonated silica gel slightly improves the yield of the axial diastereomer to ≤27%. Even for the equatorial diastereomer, the need to separate diastereomers represents a limitation for material throughput. For other TCO derivatives, chromatographic separation of diastereomers can be very difficult and is not always feasible by flash chromatography.

One solution to the diastereomer issue has been to utilize s-TCO as a highly strained dienophile prepared from the meso compound precursor. s-TCO is the most reactive dienophile known, and is suitable for applications as a probe molecule in bioorthogonal chemistry, but due to alkene isomerization, s-TCO is often unsuitable as a probe molecule where more prolonged cellular incubation is required. Currently, several TCO derivatives are available for purchase, but they are very expensive. Thus, a general and diastereoselective synthesis of TCO derivatives could greatly increase the availability of these useful compounds for chemical biology research.

Nagendrappa in Tetrahedron 1982, 38, 2429-2433, describes having produced a mixture of compound 7, trans-cyclooct-3-eneone (Nagendrappa's compound 8) and trans-cyclooct-4-eneone (Nagendrappa's compound 9). The inventors of the present application believe that in view of the harsh reaction conditions employed by Nagendrappa and based on an analysis of the listed ¹H NMR peaks, Nagendrappa did not actually form compound 9 shown below.

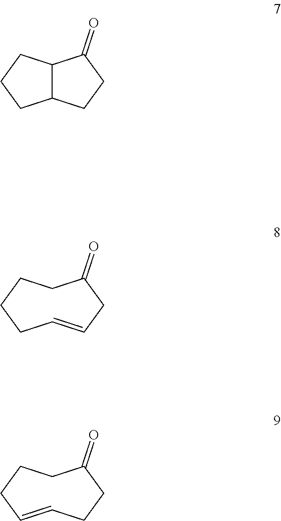 [SMILES: O=C1CCC2CCCC12 ; O=C1C/C=C/CCCC1 ; O=C1CC/C=C/CCC1]

In addition to the issue of stereoselectivity, hydrophobicity has been a longstanding issue with TCOs where non-specific binding and extensive wash-out protocols in live cell assays produce undesirable consequences. Recently, heterocyclic trans-cyclooctenes with backbone oxygen atoms have been shown to have higher hydrophilicity and improved physiochemical properties for cellular and in vivo imaging applications. However, a drawback for these oxo-TCOs is lengthy syntheses that produce diastereomers that can be separated only with difficulty.

Hence, there is a need for a new scalable method for synthesizing TCOs with favorable physiochemical properties in high yield through the stereocontrolled addition of nucleophiles to trans-cyclooct-4-enone 2 (FIG. 1B).

### SUMMARY OF THE INVENTION

As discussed hereinabove, bioorthogonal chemistry has become an essential tool for biotechnology and an emerging tool in medicine. One of the more important reagents, TCO, is difficult to prepare, and the lipophilicity of trans-cyclooctene derivatives can limit their applications in cells and in vivo. Disclosed herein is a simple method to make TCOs quickly and selectively to give derivatives with improved lipophilicity.

Disclosed herein is trans-cyclooct-4-eneone having the following formula (2):

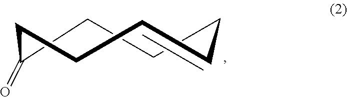

wherein the trans-cyclooct-4-eneone 2 characterized by ¹H NMR (400 MHz, CDCl₃) includes peaks at 5.27 ppm and 2.91 ppm, in accordance with the embodiments of the present inventions.

In an embodiment, the trans-cyclooct-4-enone 2 is in an isolated form. In a specific embodiment, the trans-cyclooct-4-enone 2 is at least 85% pure, or at least 90% pure, or at least 95% pure, or at least 97% pure, or at least 99% pure.

The trans-cyclooct-4-enone 2 can be produced by a photochemical flow method comprising irradiating cis-cyclooct-4-enone 1 with light from a low-pressure mercury lamp for a time sufficient to form the trans-cyclooct-4-enone.

In another aspect of the present invention, there is a substituted axial hydroxy-trans-cyclooctene, having the following formula (2a):

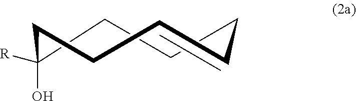

where R is selected from hydrogen, alkyl, aryl, and heteroaryl. In an embodiment, R is selected from hydrogen, allyl, acetate, cyano, acetohydrazide, hydroxyethyl, (prop-2-yn-1-yloxy)ethyl, amino ethyl, hydroxysuccinyl acetate, phenyl, phenylethynyl, and the like.

In an embodiment, the substituted axial hydroxy-trans-cyclooctene 2a exists as a single diastereoisomer. In another embodiment, the axial hydroxy-trans-cyclooctene 2a is isolated and is at least 75% pure, or at least 80% pure, or at least 85% pure, or at least 90% pure, or at least 95% pure, or at least 99% pure.

In various non-limiting embodiments, the substituted axial hydroxy-trans-cyclooctene 2a has one of the following structures:

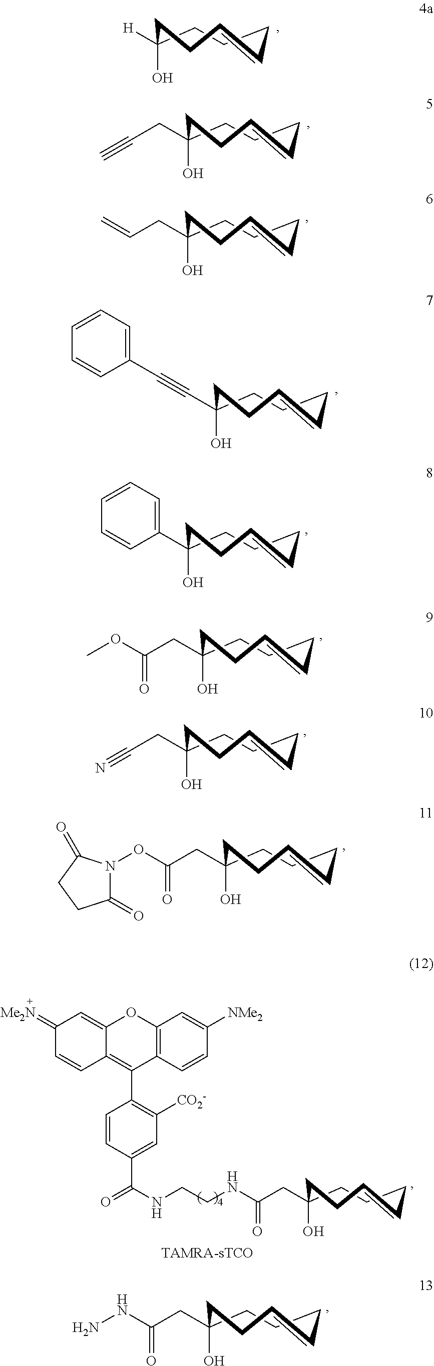

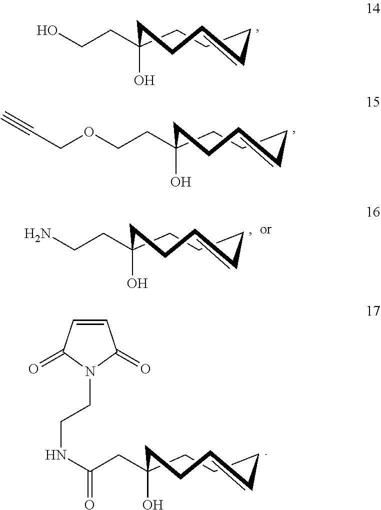

In another aspect of the present invention, there is an alpha-substituted trans-cyclooct-4-enone, having the formula:

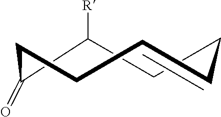

where R′ is selected from the group consisting of alkyl, aryl, carboxylic acid, alkene, and alkyne. In an embodiment, R′ is selected from methyl, benzyl, carboxylic acid, allyl, and propargyl. In various non-limiting embodiments, the alpha-substituted trans-cyclooct-4-enone has one of the following structures:

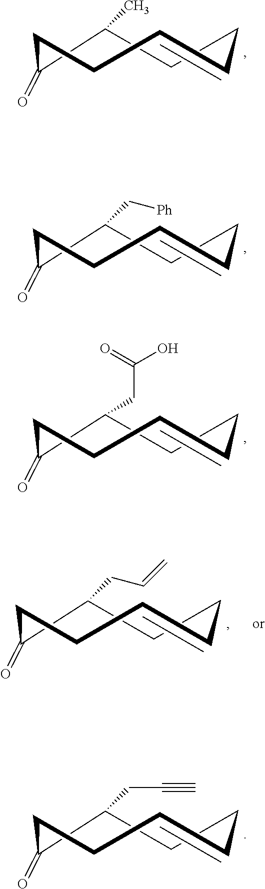

In another aspect of the invention, there is an oxime conjugate having the following formula:

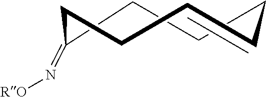

where R″ is selected from the group consisting of hydrogen, alkyl, and aryl. In various non-limiting embodiments, the oxime conjugate has one of the following structures:

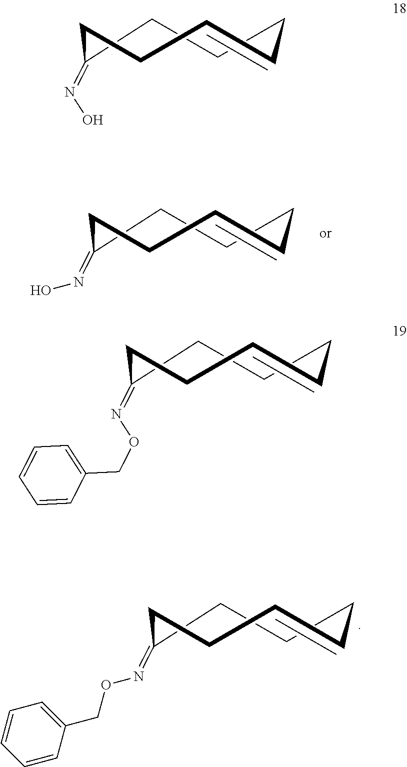

In yet another aspect, there is a method of producing the substituted axial hydroxy-trans-cyclooctene 2a. The method comprises contacting trans-cyclooct-4-enone with a nucleophile for a stereocontrolled 1,2-addition of the nucleophile to the trans-cyclooct-4-enone, such that the nucleophilic addition to the trans-cyclooct-4-eneone 2 take place exclusively from the equatorial-face of the trans-cyclooctenone to produce an axial hydroxy-trans-cyclooctene 2a as a single diastereomer. In an embodiment, nucleophile is a Grignard reagent or an organometallic such as an organolithium, and an organozinc. Suitable nucleophiles include, but are not limited to, lithium phenyl acetylene, methyl α-lithioacetate, lithioacetonitrile, lithium bis(trimethylsilyl), and the like.

In an embodiment, the method of producing the alpha-substituted trans-cyclooct-4-enone 2a may comprise treating the trans-cyclooct-4-enone 2 with a base followed by the addition of an electrophile. Suitable bases for the reaction include, but are not limited to, sodium hexamethyldisilazide, lithium hexamethyldisilazide, potassium hexamethyldisilazide, sodium hydride, lithium diisopropylamide, sodium diisopropylamide, and the like. Suitable electrophiles include, but are not limited to, alkyl halides, alkyl sulfonates, aldehydes, epoxides, aldehydes, ketones, and the like.

In an embodiment of the method using electrophilic substitution, the substituted axial hydroxy-trans-cyclooctene 2a is produced as a single diastereoisomer. In a specific embodiment, the substituted axial hydroxy-trans-cyclooctene 2a is produced with a yield of at least 80% pure, or 85%, or 90%, or 95%.

### BRIEF DESCRIPTION OF THE DRAWINGS

FIG. 1A (Prior Art) shows a common method of synthesizing TCOs.

FIG. 113 displays an exemplary diastereoselective method for synthesizing TCOs, according to an embodiment of the present invention.

FIG. 2A shows a synthesis of cis-cyclooct-4-enone 1 and trans-cyclooct-4-enone 2, according to an embodiment of the present invention.

FIG. 2B shows a reaction of trans-cyclooct-4-enone 2 with LiAlH₄. Nucleophilic addition can occur to the equatorial or axial face of 2 producing diastereomers 4a and 4b, respectively, according to an embodiment of the present invention.

FIG. 2C shows transition state calculations prediction that nucleophilic addition to equatorial face would be favored over the axial face.

FIG. 3A shows Scheme 1 depicting nucleophilic addition reactions of trans-cyclooct-4-enone (2) can serve as a universal platform for the diastereoselective synthesis of a-TCOs as well as oxime conjugates, according to an embodiment of the present invention.

FIG. 3B shows some exemplary functional derivatives readily available from conjugation precursors 9 and 10, according to an embodiment of the present invention.

FIG. 4A shows stopped flow kinetics under pseudo-first order conditions used to determine second order rate constants for the reactions of tetrazine 20 with 14, allowing comparison to less reactive 4a and 4b. (^{a}k₂ measured in 95:5 water:MeOH. ^{b}The reaction of 4a with a PEGylated amide of 20 in 100% H₂O was previously measured as k₂ 80,200 (Darko et al., Chem. Sci. 2014, 5, 3770-3776.). ^{c}k₂ previously measured with 20 in 100% H₂O (Lambert et al., Org. Biomol. Chem. 2017, 15, 6640-6644). ^{d}k₂ previously measured with PEGylated amide of 20 in 100% H₂O (Ibid, Darko et al.).

FIG. 4B shows cLogP calculations for a series of analogs of TCO, oxoTCO, d-TCO, and a-TCO to illustrate the improved hydrophilicity of a-TCO conjugates.

FIG. 4C shows cell permeability, as demonstrated by incorporation of MeTz-Halo (21) into HeLa cells transfected with either H2B-HaloTag-GFP (nuclear) or GAP43-HaloTag-GFP (cytoplasmic), followed by labeling with TAMRA-a-TCO (1 μM). Confocal microscopy images of transfected cells labeled with TAMRA-a-TCO show subcellular colocalization of GFP and TAMRA fluorescence, consistent with selective intracellular labeling. Scale bars for H2B and GAP43 labeling are 5 μM and 10 μM, respectively.

FIG. 5A shows structures of TAMRA and conjugates with TCO, oxo-TCO and a-TCO. (B, C) HeLa cells were incubated for 30 min with TAMRA-dyes, and cells were initially washed three times with PBS, and then cell media was exchanged after 10, 40 and 120 minutes. After each wash, cells were imaged live by fluorescence microscopy with illumination at 531 nm and with fixed-intensity across all samples.

FIG. 5B shows widefield images cells after 3 washes.

FIG. 5C shows comparison of background fluorescence across all experiments, quantified by dividing total fluorescence by the number of cells in each image.

FIG. 6A shows stability profiles of TCOs 14 and 4a in methanol-d₄ (35 mM) over 7 days.

FIG. 6B shows stability profiles of TCOs 14 and 4a in phosphate buffered D₂O, pD=7.4 (33 mM) over 5 days.

FIG. 6C shows stability profiles of TCOs 14 and 4a with mercaptoethanol (25 mM) in phosphate buffered D₂O (pD=7.4).

FIG. 7A shows that bimolecular rate constant was determined by the linear regression analysis of k_{obs} versus the 5-ax-hydroxy-5-eq-(2-hydroxyethyl)-trans-cyclooctene 14 final concentrations.

FIG. 7B shows that bimolecular rate constant was determined by the linear regression analysis of k_{obs} versus the 5-ax-hydroxy-trans-cyclooctene 4a final concentrations.

FIG. 8 shows TAM RA-TCO comparative washout assay. HeLa cells were incubated with 5 μM of TAMRA-TCO, TAMRA-oxo-TCO, TAMRA-a-TCO, or TAMRA for 30 minutes. The cells were washed with DPBS 3× followed by media exchanges at 10 min, 40 min, or 2-hour time intervals. Fluorescence microscopy was used to quantify background labeling at specified time intervals after exchanging with fresh media. Images were obtained on an EVOS M7000.

FIG. 9 shows an ¹H NMR spectrum of trans-cyclooct-4-enone 2 in CDCl₃. The bolded peaks were not observed by Naggendrappa (Tetrahedron, 1982, 38, 2429-2433). ¹H NMR (400 MHz, CDCl₃) δ 5.88 (ddd, J=15.5, 11.1, 3.8 Hz, 1H), 5.27 (ddd, J=15.6, 10.9, 3.8 Hz, 1H), 2.91 (ddd, J=12.6, 10.4, 6.2 Hz, 1H), 2.68-2.54 (m, 1H), 2.54-2.38 (m, 2H), 2.37-2.22 (m, 2H), 2.07-1.78 (m, 4H).

FIG. 10 (Prior Art) (a) Preparation and X-ray structure of a trans-cyclooctene with an axial substituent. 1, 3-Diaxial interactions are highlighted. (b) Stereoscopic, transannular cyclization.

### DETAILED DESCRIPTION OF THE INVENTION

As used herein, the term “alkyl group” refers to a saturated aliphatic hydrocarbon group such as a methyl group, an ethyl group, an n-propyl group, an isopropyl group, an n-butyl group, a sec-butyl group, or a tert-butyl group, and the alkyl group may have a substituent or no substituent. In an embodiment, the alkyl group is an unsubstituted alkyl. In another embodiment, the alkyl group is a substituted alkyl group. The term “substituted alkyl group” refers to an alkyl group bonded to a substituent, the additional substituent is not particularly limited. Examples of the additional substituent include an alkyl group, a halogen, an aryl group, and a heteroaryl group, and the same holds true in the description below. An alkyl group substituted with a halogen is also referred to as a haloalkyl group. The number of carbon atoms in the alkyl group is not particularly limited, and is preferably in the range of 1 to 12.

As used herein, the term “aryl group” refers to an aromatic hydrocarbon group such as a phenyl group, a biphenyl group, a naphthalene group, a terphenyl group. The aryl group may have a substituent or no substituent. In an embodiment, the aryl group is an unsubstituted aryl. In another embodiment, the aryl group is a substituted aryl group. The term “substituted aryl group” refers to an aryl group bonded to a substituent, the additional substituent is not particularly limited. An aryl group substituted with a halogen is also referred to as a haloaryl group. The number of carbon atoms in the aryl group is not particularly limited, and is preferably in the range of 6 to 14.

In a substituted phenyl group having two adjacent carbon atoms each having a substituent, the substituents may form a ring structure. The resulting group may correspond to any one or more of a “substituted phenyl group”, an “aryl group having a structure in which two or more rings are condensed”, and a “heteroaryl group having a structure in which two or more rings are condensed” depending on the structure.

As used herein, the term “heteroaryl group” refers to a cyclic aromatic group having one or a plurality of atoms other than carbon in the ring, such as a pyridyl group, a furanyl group, a thiophenyl group, a quinolinyl group, an isoquinolinyl group, a pyrazinyl group, a pyrimidyl group, a pyridazinyl group, a pyrrolyl group, an imidazolyl group, an oxazolyl group, an isoxazolyl group, a thiazolyl group, an isothiazolyl group, an indazolyl group, a benzofuranyl group, a benzothiophenyl or a triazinyl group. The heteroaryl group may have a substituent or no substituent. The number of carbon atoms in the heteroaryl group is not particularly limited, and is preferably in the range of 2 to 12.

As used herein, the term “halide” refers to an ion selected from fluoride, chloride, bromide, and iodide.

As used herein, the term “acyl group” refers to a functional group having an alkyl group, a cycloalkyl group, an alkenyl group, an alkynyl group, an aryl group, or a heteroaryl group bonded via a carbonyl group, such as an acetyl group, a propionyl group, a benzoyl group, or an acrylyl group, and these substituents may be further substituted. The number of carbon atoms in the acyl group is not particularly limited, and is 2 to 10.

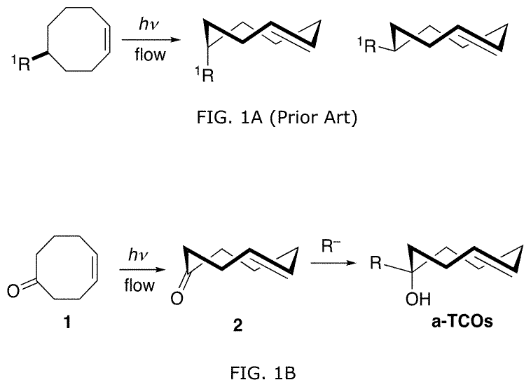

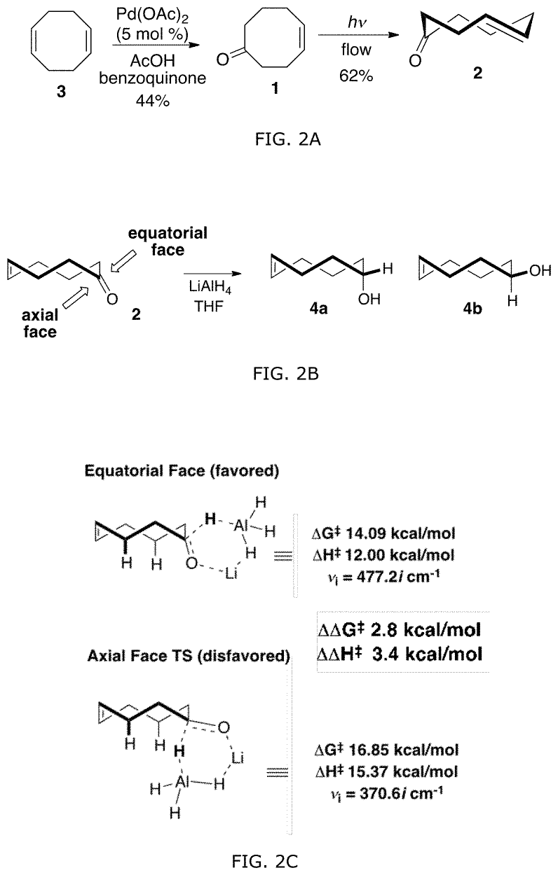

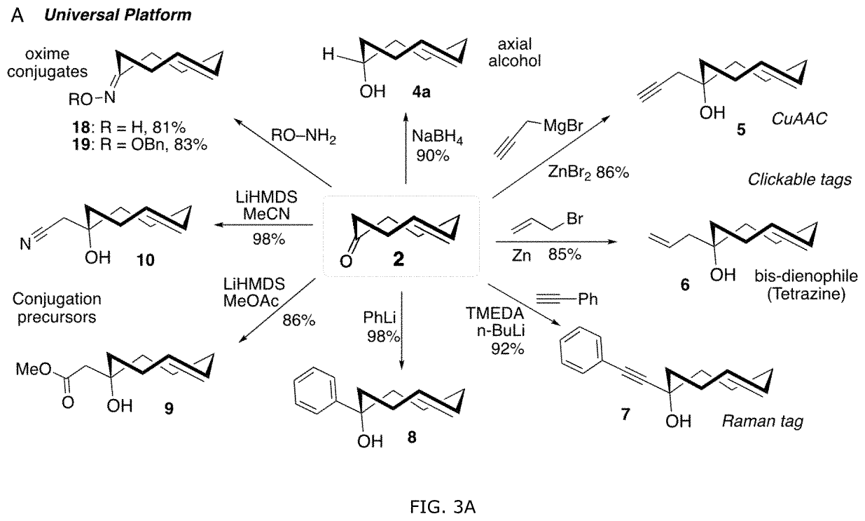

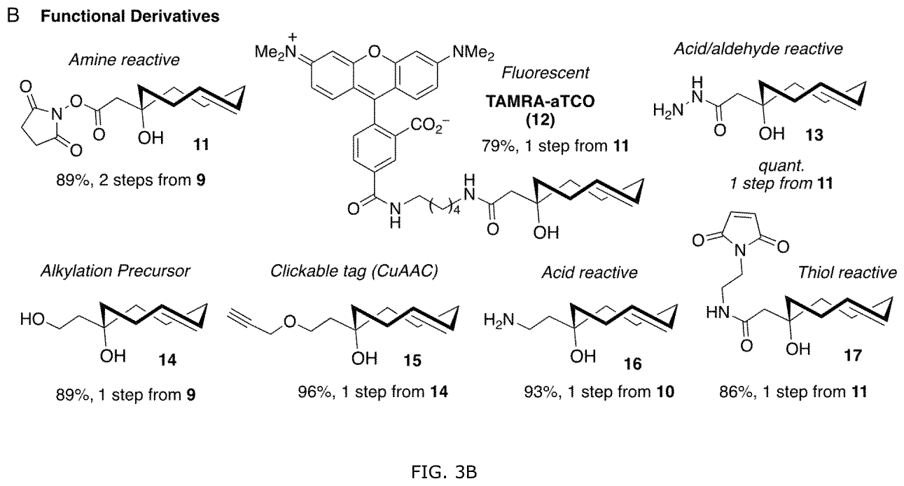

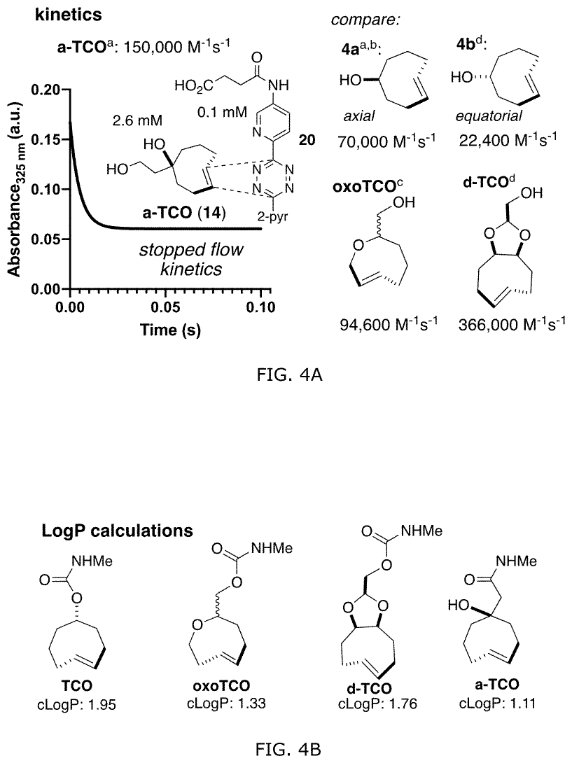

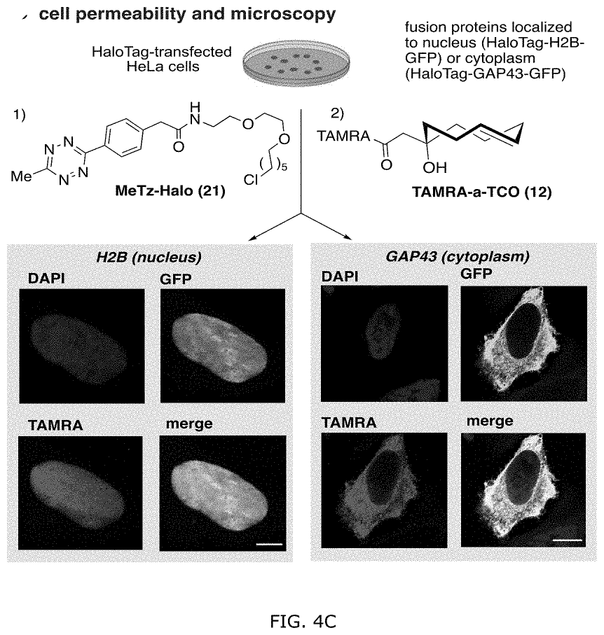

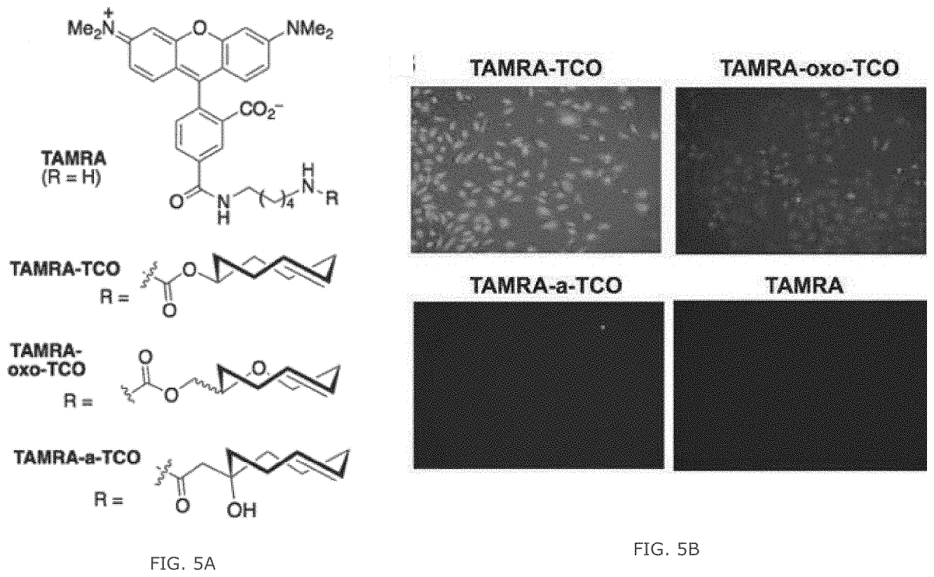

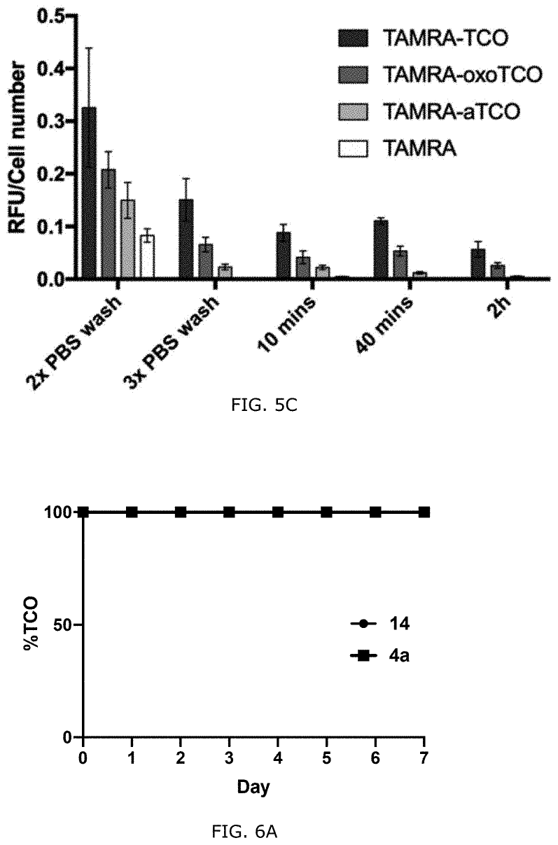

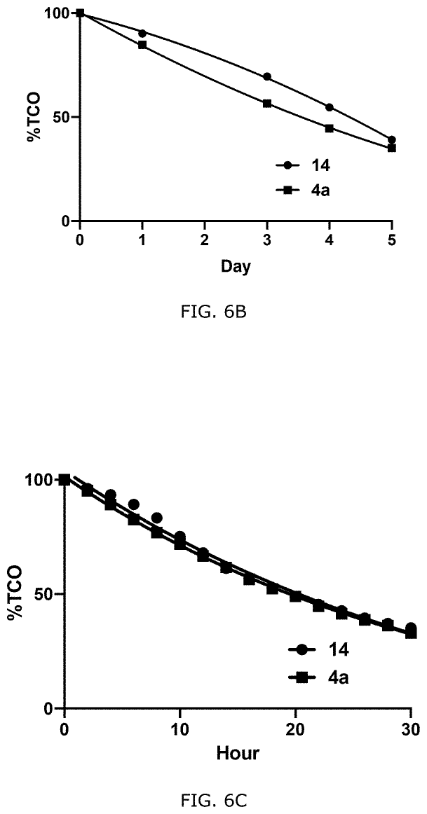

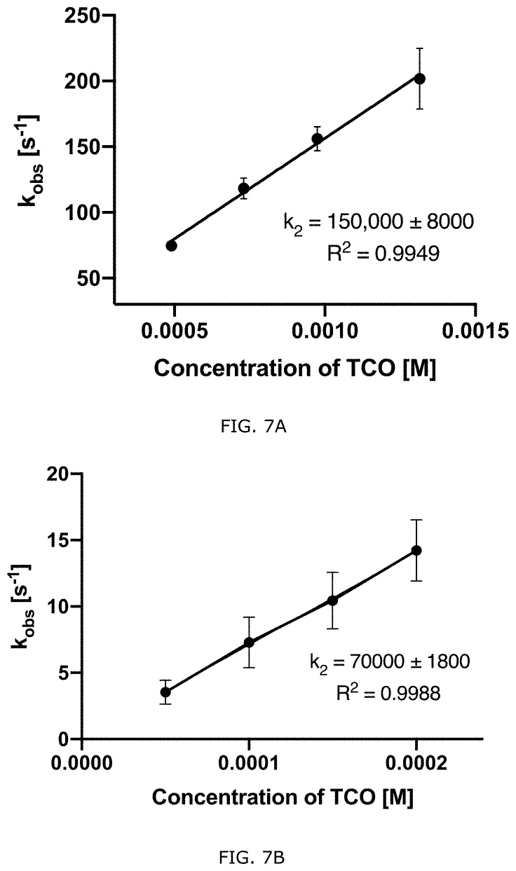

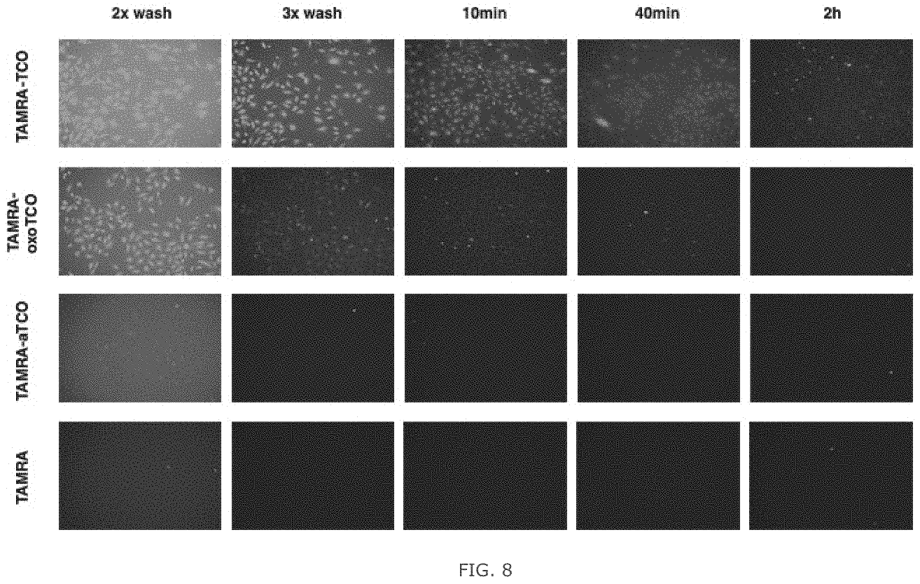

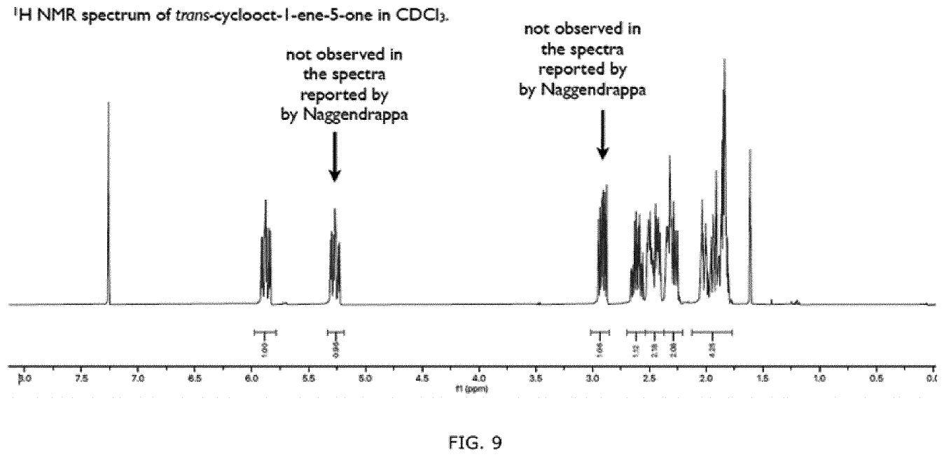

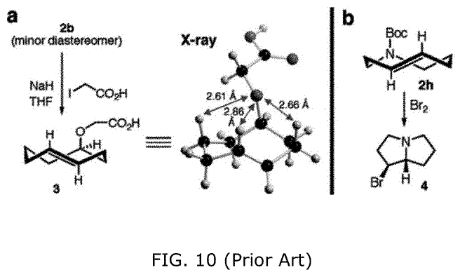

As shown in FIG. 1B, trans-cyclooct-4-enone 2 lacks a stereocenter, it therefore can be prepared on large scale using flow photochemistry from cis-cyclooct-4-enone 1 without the complication of diastereoselectivity in the photoisomerization step encountered previously. The rigid structure of 2 distinguishes the faces of the ketone, and computation was used to predict that nucleophilic additions to 2 take place exclusively from the equatorial-face of the ketone to produce axial products as single diastereomers. By engaging a range of nucleophiles, ketone 2 serves as a general platform for preparing a range of functionalized axial-5-hydroxy-trans-cyclooctene (‘a-TCO’) analogs. The method provides improved access to new compounds as well as known TCO derivatives required for in vivo radiochemistry, click-to-release chemistry, and sulfenic acid detection in live cells. An a-TCO derivative was shown to be more reactive than both axial and equatorial diastereomers of 5-hydroxy-trans-cyclooctene as well as oxo-TCO in cycloadditions with tetrazines. As a demonstration of the favorable physicochemical properties of a-TCOs, a fluorescent TAMRA derivative was shown to be cell-permeable by demonstrating intracellular Diels-Alder reactions in live cells and was shown to washout of HeLa cells more rapidly than even a hydrophilic oxo-TCO analog.

### Experiment 1

Ketone 1 was prepared simply in a single step by the Wacker oxidation (5 mol % Pd(OAc)₂, AcOH, benzoquinone) of 1,5-cyclooctadiene 3 on multigram scale (FIG. 2A). Alternately, 1 can be prepared in a single step by Dess-Martin oxidation of 5-hydroxy-cis-cyclooctene. Photochemical isomerization of 1 was conducted using the general photochemical flow method. Using a small cartridge and bed of capture silica, ketone 2 was prepared in 62% yield and at a rate of approximately 150 mg/h, and in a typical workflow, ketone 2 was prepared in 2.5 g batches.

Computation was used to predict if nucleophilic addition to TCO 2 would be diastereoselective (FIG. 2B). It was reasoned that 1,3-diaxial interactions in the lowest energy ‘crown’ conformation of the parent TCO would favor addition to the equatorial face by nucleophiles, akin to the facial selectivity of conformationally biased cyclohexanones. The barriers for the reaction of LiAlH₄ with 2 were calculated at the M06L/6-311+G(d,p) level using a SCRF solvent model for THF (FIG. 2C). The calculated barriers relative to the pre-reaction complex for the equatorial attack by hydride are ΔG‡ 14.09 kcal mol⁻¹ and ΔH‡ 12.00 kcal mol⁻¹. The barrier is significantly lower than that calculated for axial attack (ΔΔG‡ 2.8 kcal/mol and ΔΔH‡ 3.4 kcal/mol). Encouraged by these computational predictions, inventors experimentally investigated additions of nucleophiles to TCO 2 (Scheme 1 shown in FIGS. 3A-3B). In agreement with the computational data, nucleophilic addition of hydride occurred exclusively to the equatorial face of 2 to produce 4a. While LiAlH₄ gave 4a in only 67% yield due partial alkene isomerization, NaBH₄ provided 4a as a single diastereomer in 90% yield with no isomerization. Derivatives of axial alcohol 4a are especially useful for their rapid kinetics and their utility in click-to-release chemistry, but previous syntheses of 4a were low yielding (≤27%) and required separation from major diastereomer 4b. The improved route described here is short, selective and more scalable.

Nucleophilic additions with a diverse array of nucleophiles were preformed to create a library of a-TCOs (Scheme 1, shown in FIGS. 3A-3B). Compound 5 bearing a bioorthogonal alkyne tag was recently developed for the capture of cellular protein sulfenic acids via transannular thioetherification with subsequent proteomic analysis enabled by a CuAAC-chemistry workflow. Inventors note that 5 (and other a-TCOs) are still suitable for selective bioorthogonal chemistry as they only modify sulfenic acids at relatively high TCO concentration (generally ≥500 μM), but not under the conditions typically used in intracellular bioorthogonal chemistry (generally ≤10 μM). In the previous study, 5 was synthesized by direct photoisomerization in only 6% yield and required separation from two isomers. As shown in FIG. 3A, compound 5 can be prepared in 86% yield as a single diastereomer by adding propargyl magnesium bromide to ketone 2. Similarly, compound 6 bearing a simple alkene tag can be constructed in 85% yield and as a single diastereomer by the addition of allyl zinc bromide to ketone 2. Like trans-cyclooctenes, simple α-olefins can function as dienophiles in tetrazine ligation but with much slower kinetics, providing handles for potential sequential bioorthogonal chemistry applications. Organolithium nucleophiles generated in-situ were used to synthesize TCOs 7-8. Illustrating the ability to introduce a tag that may be useful for Raman spectroscopy and imaging, TCO 7 bearing a phenylacetylene group was synthesized by the addition of lithium phenyl acetylene to 2 in 92% yield. TCO 8 was synthesized similarly by the addition of phenyl lithium to 2 in 98% yield and serves as a model reaction for nucleophilic addition of aryl groups to TCO 2.

The diastereoselective additions of 2 with methyl α-lithioacetate or lithioacetonitrile provided a straightforward path to introduce handles for conjugation via amide bond or ether formation. The reaction of ketone 2 with lithium bis(trimethylsilyl)amide (LiHMDS)/methyl acetate produced ester 9 in 86% yield. Similarly, the combination of 2 with LiHMDS and acetonitrile produced nitrile 10 in 98% yield. Hydrolysis of 9 with trimethyltin hydroxide followed by DIC-mediated coupling with N-hydroxysuccinimide gave NHS ester 11 in 89% yield (FIG. 3B). Alternately, using LiOH gave 11 in 46% yield after DIC coupling. Through amide coupling, 11 gave the fluorescent TAMRA-conjugate 12 with favorable physiochemical properties, and 11 was also combined with hydrazine monohydrate to give hydrazide 13 with a potential handle for aldehyde conjugation. TCO 9 also was combined with LiAlH₄ to provide diol 14 in 89% yield which was next used in a Williamson ether synthesis with NaH/propargyl bromide to give 96% yield of propargyl ether 15. Compound 15 serves a more stable alternative to alkyne-tagged TCO 6, which is found to polymerize if not stored in solution. Amine 16 was synthesized by LiAlH₄ reduction of TCO nitrile 10 in 93% yield to introduce an acid reactive conjugation handle. Maleimide 17 was prepared though amide coupling of TCO 11 with N-(2-aminoethyl)maleimide in 86% yield. The yield of 17 is significantly higher than what was observed in previous preparations of TCO-maleimides from activated nitrophenylcarbonates. Furthermore, oxime conjugates 18 and 19 could be prepared by the direct condensation of the corresponding hydroxylamines in the presence of pyridine (FIG. 3A). In summary, ketone 2 can serve as a readily prepared, central intermediate for the diastereoselective preparation of a range of hydrophilic a-TCO conjugates.

a-TCO derivatives also display rapid kinetics in Diels-Alder reactions compared to most other TCOs. The kinetics for the reaction of a-TCO derivative 14 toward a 3,6-dipyridyl-s-tetrazinyl succinamic acid derivative 20 were measured by stopped flow kinetics at 25° C. in 95:5 PBS:MeOH (FIG. 4A). With a second-order rate constant of 150,000±8000 M⁻¹s⁻¹, a-TCO is more than twice as reactive toward 20 as the axial isomer of 5-hydroxy-trans-cyclooctene 4a (70,000±1800 M⁻¹s⁻¹), and nearly 7-times more reactive than equatorial isomer 4b (22,400±40 M⁻¹s⁻¹). The faster kinetics are likely due an increase in olefinic strain for a-TCO due to steric effect of geminal substitution in the 8-membered ring backbone. Likely for the same reason, similar rate accelerations have been observed for more sterically encumbered derivatives of 4a. a-TCO 14 is also more reactive than oxo-TCO, and the conformationally strained, bicyclic d-TCO is only 2.2-times more reactive than 14. As shown in FIG. 4B, a-TCO derivatives are also calculated to have improved physiochemical properties relative to other TCO derivatives. While both oxo-TCO and d-TCO were previously introduced as less hydrophobic bioorthogonal reagents, the methylamine conjugate of a-TCO is calculated to have even a lower cLogP value.

The stability of a-TCO 14 is very similar to that of axial-5-hydroxy-trans-cyclooctene 4a, which is used broadly for applications in bioorthogonal chemistry. In MeOD, 35 mM solutions of both 14 and 4a are >99% stable after 1 week at room temperature (FIG. 6A). After 24 h at room temperature in D₂O—PBS (pD 7.4), 33 mM solutions of 14 and 4a display 90% and 85% stability, respectively. In D₂O—PBS containing 25 mM mercaptoethanol, 49% of both 14 and 4 remained after 20 h.

The fluorescent conjugate TAMRA-a-TCO 12 was shown to be cell permeable through selective bioorthogonal reaction inside live cells using the HaloTag self-labeling platform (FIG. 4C). Here, cells are transfected with a GFP-HaloTag construct fused to a protein that controls subcellular localization, and then labeled by a tetrazine-HaloTag ligand 21. Upon subsequent reaction with fluorescent TAMRA-a-TCO, conjugation is expected only in those cells that express the HaloTag fusion protein, and co-localization of GFP and TAMRA fluorescence is expected. As shown in FIG. 4C, HeLa cells were transfected with either HaloTag-H2B-GFP (nucleus) or HaloTag-GAP43-GFP (cytoplasm), labeled with MeTz-Halo 21 (10 μM), washed and then treated with TAMRA-a-TCO (1 μM) for 30 min, at which point the TCO reagent was chased by a non-fluorescent tetrazine, and the cells were fixed and imaged. As shown in FIG. 4C, for both nuclear and cytoplasmic targets, selective colocalization of the TAMRA signal with GFP was observed in both cases.

The advantage of the increased hydrophilicity for a-TCO conjugates was demonstrated by comparing the washout times for fluorescent derivatives in mammalian cells. While TCO-derivatives offer rapid labeling kinetics, a consideration for labeling by fluorophore-tagged TCOs has been the background fluorescence due to non-covalent cellular binding that can be ameliorated only by extended washout times. More hydrophilic oxo- and dioxo-trans-cyclooctenes with backbone oxygens offer improvements, but are difficult to synthesize and, for dioxo-TCO, display reduced Diels-Alder kinetics. As shown in FIGS. 5A-5C, the fluorescent conjugates TAM RA-TCO, TAMRA-oxo-TCO and TAMRA-a-TCO were prepared and compared their cellular washout times to unconjugated TAMRA. Thus, HeLa cells were incubated for 30 min with TAMRA-dyes, and cells were initially washed three times with DPBS, and then cell media was exchanged after 10, 40 and 120 minutes. After each wash, cells were imaged live by fluorescence microscopy with illumination at 531 nm and fixed-intensity across all samples. Widefield images of the cells after 3 washes are shown in FIG. 5B; images after the earlier and later washings are shown in FIG. 8. Background fluorescence was quantified by dividing total fluorescence by the number of cells in each image (FIG. 5C). For TAM RA-TCO, cells are markedly fluorescent after 3 washings, and still display significant background after washing for 2 hours. The background is improved with TAMRA-oxo-TCO and especially with TAMRA-a-TCO, which after initial 3× wash shows an 85% reduction in background fluorescence relative to TAMRA-TCO. After 2 hours, washout of TAMRA-a-TCO is essentially complete with background equivalent to TAMRA itself, whereas TAM RA-TCO and TAMRA-oxo-TCO both still display residual fluorescence even after 2 hours.

In summary, a-TCOs are a class of trans-cyclooctenes with favorable physiochemical properties that can be prepared in high yield through the stereocontrolled additions of nucleophiles to trans-cyclooct-4-enone (2), a trans-cyclooctene that can be prepared on large scale in two steps from 1,5-cyclooctadiene. Computation was used to rationalize diastereoselectivity of 1,2-additions to deliver a-TCO products. The strategy can be applied to the synthesis of a range of usefully functionalized a-TCOs with high yield, selectivity. a-TCOs were also shown to be more reactive than standard TCOs and less hydrophobic than even hydrophilic oxo-TCO analogs. As a demonstration of the favorable physicochemical properties of a-TCOs, a fluorescent TAMRA derivative was shown to be cell-permeable by demonstrating intracellular Diels-Alder chemistry in live cells and to washout of HeLa cells more rapidly and completely than TCO and oxo-TCO analogs.

### Experiment 2

Alkylation of 2 was carried out to produce an alpha-substituted trans-cyclooct-4-ene 20 with a variety of R groups, including but not limited to alkyl, benzylic, carboxylic acid, alkene, and alkyne. This included reaction of 2 with LiHMDS followed by addition of an alkylhalide electrophile.

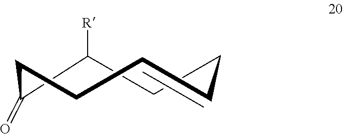

### Experiment 3

¹H NMR (400 MHz) spectrum of trans-cyclooct-4-eneone 2 in CDCl₃ was taken and peaks were compared with those reported by Nagendrappa in Tetrahedron 1982, 38, 2429-2433. As shown in FIG. 9, most strikingly, there are two diagnostic peaks at 5.27 ppm and 2.91 ppm in the spectrum of trans-cyclooct-4-eneone 2 of the present invention, that are absent in the data reported by Nagendrappa.

Additionally, the structure of trans-cyclooct-4-eneone 2 of the present invention was unambiguously confirmed by converting 2 into axial-5-hydroxy-trans-cyclooctene, which is well known, commercially available, and has been converted into a crystallographically characterized derivative as described in FIG. 2 of Maksim Royzen, Glenn P. A. Yap, and Joseph M. Fox Journal of the American Chemical Society 2008 130 (12), 3760-3761, reproduced here as FIG. 10. It should be noted that J. Am. Chem. Soc. 2008, 130, 3760-3761 has been cited 130 times according to ACS, and that axial-5-hydroxy-trans-cyclooctene prepared by the procedure described in J. Am. Chem. Soc. 2008, 130, 3760-3761 has been used in the literature, e.g., Rossin et al., Highly Reactive trans-Cyclooctene Tags with Improved Stability for Diels-Alder Chemistry in Living Systems, Bioconjugate Chemistry 2013, 24 (7), 1210-1217; and Arcadio et al., Mechanism-Based Fluorogenic trans-Cyclooctene-Tetrazine Cycloaddition, Angewandte Chemie International Edition 2017, 56, 1334-1337.

Additionally, inventors also took the spectrum of both axial-5-hydroxy-trans-cyclooctene and equatorial-5-hydroxy-trans-cyclooctene, and compared to the spectral report by Nagendrappa. The spectra differ, but due to a large spectral window for the impure mixture of Nagendrappa, it is not clear if axial-5-hydroxy-trans-cyclooctene was a component of their mixture.

### ASPECTS OF THE INVENTION

Certain illustrative, non-limiting aspects of the invention may be summarized as follows:
Aspect 1. A trans-cyclooct-4-eneone having the following formula (2):

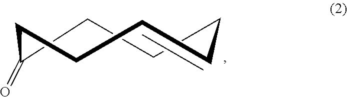

wherein the trans-cyclooct-4-eneone characterized by ¹H NMR (400 MHz, CDCl₃) includes peaks at 5.27 ppm and 2.91 ppm.
Aspect 2. The trans-cyclooct-4-enone of Aspect 1, wherein the trans-cyclooct-4-enone is in an isolated form.
Aspect 3. The trans-cyclooct-4-enone of Aspect 1, wherein the trans-cyclooct-4-enone is at least 90% pure.
Aspect 4. The trans-cyclooct-4-enone of Aspect 1, produced by a photochemical flow method comprising irradiating cis-cyclooct-4-enone with light from a low-pressure mercury lamp for a time sufficient to form the trans-cyclooct-4-enone.
Aspect 5. A substituted axial hydroxy-trans-cyclooctene, having the following formula (2a):

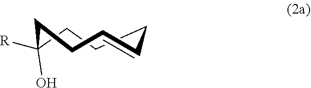

where R is selected from hydrogen, alkyl, aryl, and heteroaryl.
Aspect 6. The substituted axial hydroxy-trans-cyclooctene of Aspect 5, wherein R is selected from hydrogen, allyl, acetate, cyano, acetohydrazide, hydroxyethyl, (prop-2-yn-1-yloxy)ethyl, amino ethyl, hydroxysuccinyl acetate, phenyl, and phenylethynyl.
Aspect 7. The substituted axial hydroxy-trans-cyclooctene of Aspect 5, wherein the trans-cyclooctene exists as a single diastereoisomer.
Aspect 8. The substituted axial hydroxy-trans-cyclooctene of Aspect 5 having one of the following structures:

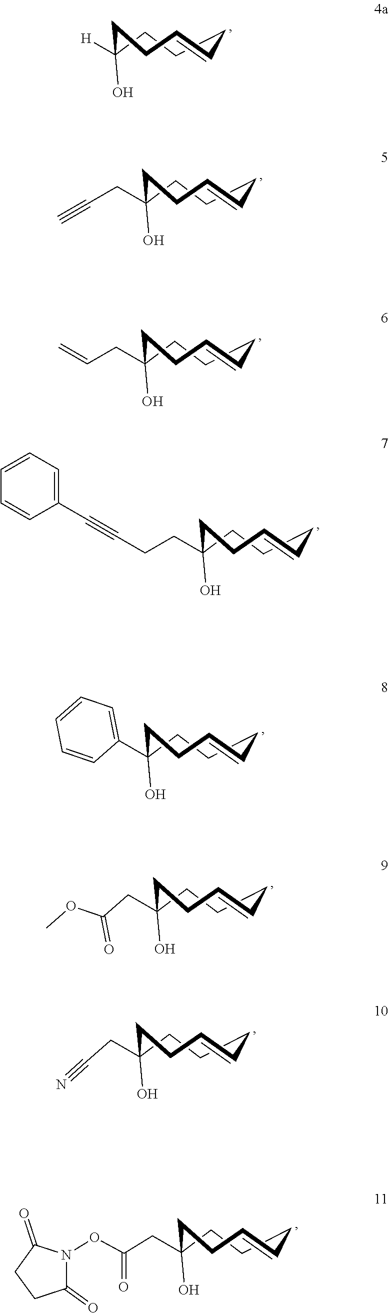

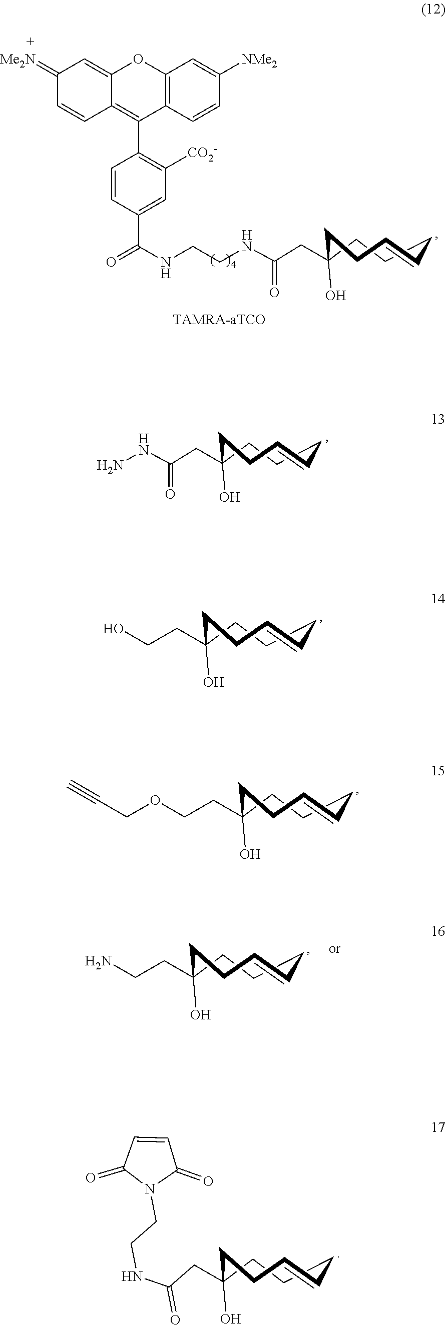

Aspect 9. An alpha-substituted trans-cyclooct-4-enone, having the formula:

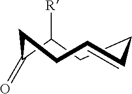

where R′ is selected from the group consisting of alkyl, aryl, carboxylic acid, alkene, and alkyne.
Aspect 10. An oxime conjugate having the following formula:

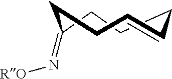

where R″ is selected from the group consisting of hydrogen, alkyl, and aryl.
Aspect 11. The oxime conjugate of Aspect 10 having one of the following structures:

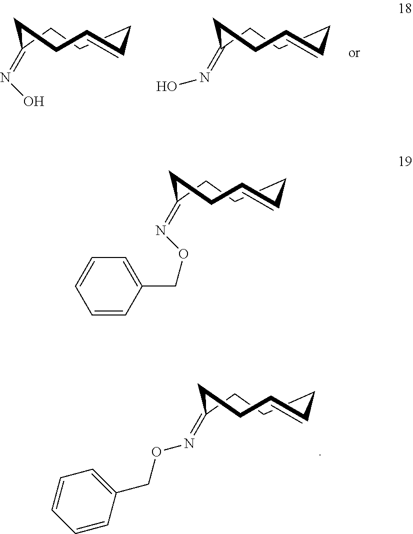

Aspect 12. A method of producing the substituted axial hydroxy-trans-cyclooctene of Aspect 5, the method comprising contacting trans-cyclooct-4-enone with a nucleophile for a stereocontrolled 1,2-addition of the nucleophile to the trans-cyclooct-4-enone, wherein nucleophilic addition to the trans-cyclooct-4-eneone take place exclusively from the equatorial-face of the trans-cyclooctennone to produce an axial hydroxy-trans-cyclooctene as a single diastereomer, and wherein the nucleophile is a Grignard reagent, an organolithium, or an organozinc.
Aspect 13. The method of Aspect 12, wherein the nucleophile is selected from lithium phenyl acetylene, methyl α-lithioacetate, lithioacetonitrile, and lithium bis(trimethylsilyl)amide.
Aspect 14. The method of Aspect 12, wherein the substituted axial hydroxy-trans-cyclooctene is produced as a single diastereoisomer.
Aspect 15. The method of Aspect 12, wherein the substituted axial hydroxy-trans-cyclooctene is produced in a yield of at least 80%.
Aspect 16. The method of Aspect 12, wherein the substituted axial hydroxy-trans-cyclooctene is at least 95% pure.
Aspect 17. A method of producing the alpha-substituted trans-cyclooct-4-enone of Aspect 9 comprising treating the trans-cyclooct-4-enone of Aspect 1 with a base followed by the addition of an electrophile.
Aspect 18. The method of Aspect 17, where the electrophile is selected from alkyl halides, alkyl sulfonates, epoxides, aldehydes, or ketones.
Aspect 19. The method of Aspect 17, wherein the substituted axial hydroxy-trans-cyclooctene is produced as a single diastereoisomer.
Aspect 20. The method of Aspect 19, wherein the substituted axial hydroxy-trans-cyclooctene is produced with a yield of at least 80%.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

### EXAMPLES

Examples of the present invention will now be described. The technical scope of the present invention is not limited to the examples described below.

Materials

Materials and their source are listed below:

Anhydrous methylene chloride, diethyl ether, and THF were obtained from an alumina column solvent purification system. Other reagents were purchased from commercial sources and used without further purification. 3-Methyl-6-(4-aminomethylphenyl)-s-tetrazine was purchased from Click Chemistry Tools.

Methods

Chromatography

Normal phase silica gel chromatography was performed on Silicycle Siliaflash P60 silica gel (40-63 μm, 60 Å) and reverse phase chromatography on prepacked Yamazen Universal Column C18-silica gel (40-60 μm, 120 Å) using automated chromatography (Teledyne Isco Combiflash Rf).

NMR Spectrometry

NMR spectra were obtained on a Bruker AV400 (¹H: 400 MHz, ¹³C: 101 MHz) and AV600 (¹H: 600 MHz, ¹³C: 150 MHz) instruments. Chemical shifts (δ) were reported in ppm and referenced according to the residual nondeuterated solvent peak: CDCl₃ (7.26 ppm), benzene-d₆ (7.16 ppm), MeOD (3.31 ppm), and DMSO-d₆ (2.50 ppm) for ¹H NMR, and CDCl₃ (77.0 ppm), benzene-d₆ (128.0 ppm), MeOD (49.0 ppm), and DMSO-d₆ (39.5 ppm) for ¹³C NMR. Coupling constants (3) were reported to the nearest 0.1 Hz for the ¹H NMR spectra and the ¹³C NMR resonances were proton decoupled. Peak multiplicities were reported as singlet (s), doublet (d), triplet (t), quartet (q), pentet (pent), multiplet (m), ‘broad’ (br), and ‘apparent’ (app). An APT pulse sequence was used for ¹³C NMR, where the secondary (CH₂) and quaternary (C) carbons appeared ‘up’, and tertiary (CH₃) and primary (CH) carbons appeared ‘down’. Exceptions were for methine carbons of alkynes, which usually have the same phase as ‘normal’ methylene and quaternary carbons.

pD and pH Measurements

Phosphate buffered D₂O (pD=7.4) was prepared by combining anhydrous sodium dihydrogen phosphate (NaH₂PO₄, 77.1 mg) and anhydrous disodium hydrogen phosphate (Na₂HPO₄, 204.4 mg) with 20 mL of D₂O to make a 0.1 M solution. The pD was measured on a Fisher Scientific AB15 Plus pH meter and pH values were converted to pD by adding 0.4 units. The pD was adjusted to 7.4 with DCI (35 wt. % in D₂O) and NaOD (40 wt. % in D₂O) as necessary.

Stopped-Flow Kinetics Measurements

Stopped-flow kinetics measurements were performed on a SX18MV-R stopped-flow spectrophotometer (Applied Photophysics Ltd.) with temperature control (25° C.).

Mass Spectrometry

Mass spectrometry was conducted on a Waters GCT Premier and Thermo Q-Exactive Orbitrap.

### GENERAL CONSIDERATIONS EXPERIMENTAL PROCEDURES

All reactions were conducted under a nitrogen atmosphere with glassware that was flame-dried under vacuum.

For purposes of long-term storage, trans-cyclooctene derivatives that are oils were stored as solutions in Et₂O in a −20° C. freezer. cLogP calculations were carried out using ALOGPS 2.1 program (available online from Virtual Computational Chemistry Laboratory).

Photoisomerization Apparatus

A previously described photoisomerization protocol was utilized with some modification. A Southern New England Ultraviolet Company Rayonet® reactor (model RPR-100 or RPR-200) was stocked with 8 low-pressure mercury lamps (2537 Å), and a 500 mL quartz flask (Southern New England Ultraviolet Company) containing the reaction solution was suspended in the reactor. A Biotage® SNAP cartridge (‘50 g’) was used to house silica gel and AgNO₃-silica gel. The bottom of the column was interfaced to PTFE tubing (⅛″ OD×0.063″ ID, flanged with a thermoelectric flanging tool), equipped with flangeless nylon fittings (¼-28 thread, IDEX part no. P-582), using a female luer (¼-28 thread, IDEX part no. P-628). The top of the column was interfaced using a male luer (¼-28 thread, IDEX part no. P-675). A fluid metering pump (Fluid Metering, Inc, model RP-D equipped with pumphead FMI R405) was interfaced to the PTFE tubing (IDEX, part no. U-510) and was used for recirculating the reaction solution through the photolysis apparatus.

Silver Nitrate Silica Gel

Flash silica gel (90 g, Silicycle, cat #R12030B, 60 Å) was suspended in 100 mL of water in a 2 L round bottomed flask. The flask was covered with aluminum foil and a silver nitrate (10 g) solution in water (10 mL) was added. The resulting mixture was thoroughly mixed. Water was evaporated under reduced pressure via rotary evaporation (bath temperature ˜65° C.) using a bump trap equipped with a coarse fritted disk. To remove the remaining traces of water, toluene (2×200 mL) was added and subsequently concentrated via rotary evaporation. The 10% silver nitrate adsorbed on silica gel was dried under vacuum overnight at room temperature then was stored in a dry, dark place.

### Keto-TCO Synthesis

### Example 1: (Z)-Cyclooct-4-enone (1)

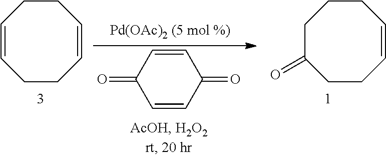

(Z)-Cyclooct-4-enone (1) can be prepared by the oxidation of commercially available 5-hydroxy-cis-cyclooctene (Combi-Blocks, QB-7357) with Dess-Martin reagent. Alternatively, 1 can be prepared from 1,5-cyclooctadiene as described below.

A round bottom flask with a magnetic stir bar was charged with Pd(OAc)₂ (1.04 g, 4.62 mmol), benzoquinone (1.01 g, 9.27 mmol), acetic acid (462 mL), 1,5-cyclooctadiene (11.3 mL, 92.4 mmol), and hydrogen peroxide (30% in H₂O, 15.7 mL, 138 mmol). After stirring for 20 hours at room temperature, the reaction mixture was diluted with 2 L of 1:1 ether/pentane which was then washed with H₂O (4×500 mL) and 8 M NaOH (4×350 mL). All aqueous washes were chilled to 0° C., combined, then stirred for 15 minutes at 0° C. The combined aqueous washes were brought to room temperature and then were extracted with 1:1 ether/pentane until all product was removed from the aqueous phase (TLC monitored). The combined organic extracts were dried with MgSO₄, filtered, and then concentrated by rotary evaporation. The residue was purified by silica gel chromatography (0-3% diethyl ether in hexanes) to afford 5.03 g (40.5 mmol, 44% yield) of a pale-yellow oil. NMR spectra were in agreement with previously reported data.

### Example 2: (E)-Cyclooct-4-enone (2)

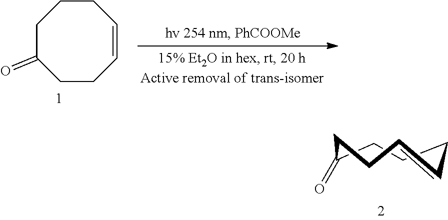

(Z)-Cyclooct-4-enone (4.00 g, 32.2 mmol), methyl benzoate (8.80 g, 64.6 mmol), and 400 mL of 15% Et₂O in hexanes were added to a 500 mL quartz flask. The flask was placed in a Rayonet® reactor containing 8 low-pressure mercury lamps (2537 Å). and connected via PTFE tubing to a column (Biotage® SNAP, 50 g) and an FMI pump. Five Biotage® SNAP cartridge (‘50 g’) columns were each packed with 7 cm of dry silica gel and topped with 16.3 g of 10% silver nitrate silica gel. The FMI pump was set at a flow rate of 100 mL/minute and the first column was flushed with 400 mL of 15% Et₂O in hexanes. The contents of the quartz flask were irradiated for 4 hours under continuous flow, after which the column was flushed with 20% Et₂O in hexanes and dried by a stream of compressed air. The flushed contents were concentrated by rotary evaporation and the recovered starting material and methyl benzoate were added back into the quartz flask. The next column was connected to the tubing and the process was repeated for each column.

After flushing the fifth column, the 10% silver nitrate silica gel from all of the columns was combined. The contents were stirred in 400 mL of ammonium hydroxide and 400 mL of CH₂Cl₂ for 10 minutes. The silica gel was filtered off and the filtrate was transferred to a separatory funnel. The aqueous layer was extracted with CH₂Cl₂ then the combined organic phases were washed with water and brine. The organics were next dried with Na₂SO₄, filtered, and concentrated by rotary evaporation in a 10° C. water bath. The crude oil was purified by silica gel chromatography (0-5% Et₂O in pentane) to afford 2.5 g (20.1 mmol, 62.5% yield) of the title compound as a pale-yellow oil. The product was stored as a 0.2 M solution in Et₂O at −20° C. ¹H NMR (400 MHz, C₆D₆) δ 5.43 (ddd, J=15.3, 11.1, 3.6 Hz, 1H), 5.21 (ddd, J=15.7, 11.2, 3.6 Hz, 1H), 2.52-2.37 (m, 1H), 2.35-2.23 (m, 1H), 2.23-2.15 (m, 1H), 2.09-1.98 (m, 2H), 1.91-1.75 (m, 1H), 1.73-1.50 (m, 3H), 1.47-1.38 (m, 1H). ¹³C NMR (101 MHz, C₆D₆) δ 214.2 (C), 133.8 (CH), 131.9 (CH), 49.1 (CH₂), 43.2 (CH₂), 34.6 (CH₂), 33.5 (CH₂), 28.3 (CH₂). FTMS (ESI+) calculated [M+H]⁺ for C₈H₁₃O 125.0966; found 125.0961.

Additionally, ¹H NMR spectrum of trans-cyclooct-4-enone 2 in CDCl₃ was taken and peaks were compared with those reported by Nagendrappa in Tetrahedron 1982, 38, 2429-2433. As shown in FIG. 9, the bolded peaks were not observed by Nagendrappa. ¹H NMR (400 MHz, CDCl₃) δ 5.88 (ddd, J=15.5, 11.1, 3.8 Hz, 1H), 5.27 (ddd, J=15.6, 10.9, 3.8 Hz, 1H), 2.91 (ddd, J=12.6, 10.4, 6.2 Hz, 1H), 2.68-2.54 (m, 1H), 2.54-2.38 (m, 2H), 2.37-2.22 (m, 2H), 2.07-1.78 (m, 4H).

### A-TCO Syntheses

### Example 3: 5-ax-Hydroxy-trans-cyclooctene (4a)

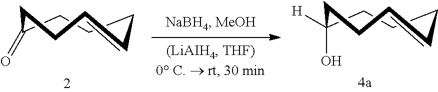

Procedure 1: (E)-Cyclooct-4-enone (104 mg, 0.837 mmol) and 1.6 mL of anhydrous MeOH were added to a round bottom flask equipped with a magnetic stir bar. The reaction mixture was cooled in an ice bath, and then NaBH₄ (66 mg, 1.7 mmol) was added in portions over the course of 10 minutes while stirring vigorously. The reaction mixture was removed from the ice bath and then stirred for 20 minutes before quenching with 1.2 mL of water at 0° C. The reaction mixture was brought to room temperature, extracted with ether, dried with Na₂SO₄, filtered, and concentrated by rotary evaporation. The crude oil was purified by silica gel chromatography (0-8% Et₂O in hexanes) to afford 95.1 mg (0.754 mmol, 90% yield) of the title compound as a colorless oil.

Procedure 2: To a flame-dried, nitrogen-purged flask was added (E)-cyclooct-4-enone (31.4 mg, 0.253 mmol) in THF (2.5 mL). The flask was placed in an ice bath and LiAlH₄ (12.5 mg, 0.329 mmol) was added in one portion. The mixture was allowed to stir at room temperature for 30 minutes, after which the flask was placed in an ice bath and the reaction mixture was quenched with water. The reaction solution was extracted with CH₂Cl₂, dried over MgSO₄, filtered, and concentrated via rotary evaporation. The crude oil was purified by silica gel column chromatography (20% Et2O in hexanes) to afford 21.3 mg (0.169 mmol, 67% yield) of the title compound as a colorless oil.

NMR spectra were in agreement with previously reported data.

### Example 4: 5-ax-Hydroxy-5-eq-propargyl-trans-cyclooctene (5)

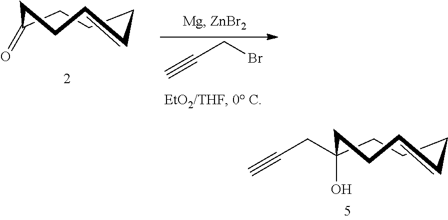

Propargyl magnesium bromide was synthesized according to a previously published procedure. Zinc bromide (140 mg, 0.621 mmol) and ground magnesium turnings (650 mg, 26.7 mmol) were added to a round bottom flask that was then thoroughly flame dried under vacuum. The flask was then charged with Et₂O (10 mL) and stirred vigorously. A solution of propargyl bromide (1.0 mL, 13 mmol) in 8 mL of Et₂O was added dropwise at room temperature until the reaction initiated, after which it was chilled to 0° C. while the remaining solution was added at a flow rate of 13.5 mL/min. The reaction mixture was stirred at 0° C. for an additional hour and formed a light green supernatant.

A round bottom flask with a magnetic stir bar was flame dried and nitrogen purged. (E)-Cyclooct-4-enone (100 mg, 0.805 mmol) and 8 mL of dry THF were added and the flask was cooled by an ice bath. The propargyl magnesium bromide solution (3 mL) was added dropwise to the flask by syringe. The reaction was monitored by TLC and then quenched with 2.5 mL of saturated NH₄Cl aqueous solution after 25 minutes. The aqueous phase was extracted with Et₂O and then the combined organic phases were dried with MgSO₄, filtered, and concentrated by rotary evaporation. The compound was purified by silica gel chromatography (0-4% ether in hexanes) to afford 113 mg (0.690 mmol, 86% yield) of the title compound as a colorless oil. ¹H NMR (400 MHz, C₆D₆) δ 5.68 (ddd, J=15.1, 10.6, 3.8 Hz, 1H), 5.40 (ddd, J=15.5, 11.3, 3.3 Hz, 1H), 2.39 (app dtd, J=12.3, 11.3, 4.5 Hz, 1H), 2.24-2.10 (m, 1H), 2.06-1.92 (m, 3H), 1.92-1.82 (m, 1H), 1.78-1.54 (m, 5H), 1.49 (ddd, J=14.0, 12.8, 4.9 Hz, 1H), 1.35 (s, 1H), 1.21 (app dd, J=15.6, 11.5 Hz, 1H). ¹³C NMR (101 MHz, C₆D₆) δ 134.7 (CH), 132.3 (CH), 81.5 (C), 71.7 (C), 71.3 (CH), 47.3 (CH₂), 39.1 (CH₂), 38.9 (CH₂), 34.4 (CH₂), 30.8 (CH₂), 27.8 (CH₂). FTMS (ESI+) calculated [M+H]⁺ for C₁₁H₁₇O, 165.1279; found 165.1271.

### Example 5: 5-eq-Allyl-5-ax-hydroxy-trans-cyclooctene (6)

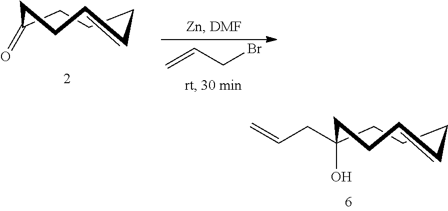

An oven-dried 4 mL vial equipped with a magnetic stir bar was charged with (E)-Cyclooct-4-enone (100 mg, 0.805 mmol), allyl bromide (99 μL, 1.17 mmol), DMF (800 μL), and 20 mesh zinc (79.0 mg, 1.21 mmol). The contents were stirred vigorously at room temperature to initiate the reaction which was indicated by a color change to brown (˜10 minutes). The reaction was monitored by TLC and quenched with saturated NH₄Cl aqueous solution 20 minutes after initiation. The aqueous phase was extracted with Et₂O then the combined extracts were dried with Na₂SO₄, filtered, and concentrated via rotary evaporation. The crude product was purified by silica gel chromatography (0-4% Et₂O in hexanes) to afford 113 mg (0.681 mmol, 85% yield) of the title compound as a colorless oil. ¹H NMR (400 MHz, C₆D₆) δ 5.75 (ddt, J=17.3, 10.2, 7.3 Hz, 1H), 5.61 (ddd, J=15.9, 10.5, 3.7 Hz, 1H), 5.45 (ddd, J=15.9, 11.2, 3.3 Hz, 1H), 5.03 (dm, J=10.1 Hz, 1H), 4.96 (dm, J=17.2 Hz, 1H), 2.37 (app qd, J=11.8, 4.7 Hz, 1H), 2.25-2.10 (m, 1H), 2.05-1.90 (m, 3H), 1.85-1.52 (m, 5H), 1.40 (ddd, J=14.0, 12.8, 4.8 Hz, 1H), 1.18-1.03 (m, 1H), 0.88 (s, 1H). ¹³C NMR (101 MHz, C₆D₆) δ 134.9 (CH), 134.2 (CH), 133.1 (CH), 118.3 (CH₂), 71.8 (C), 53.6 (CH₂), 48.0 (CH₂), 39.0 (CH₂), 34.6 (CH₂), 30.9 (CH₂), 27.8 (CH₂). FTMS (ESI+) calculated [M+H]⁺ for C₁₁H₁₉O 167.1436; found 167.1427.

### Example 6: 5-ax-Hydroxy-5-eq-phenylethynyl-trans-cyclooctene (7)

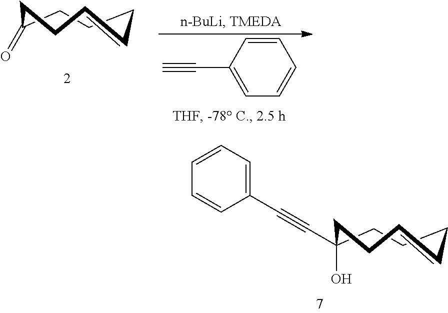

Phenyl acetylene (88 μL, 0.80 mmol) and 2 mL of THF were added to a round bottom flask with a magnetic stir bar and was then cooled by a bath of dry ice/acetone. n-Butyllithium (350 μL, 2.5 M in hexane) was added dropwise followed by TMEDA (121 μL, 0.806 mmol) and the mixture was stirred for 1 hour at −78° C. (E)-Cyclooct-4-enone (50 mg, 0.403 mmol) in 50 μL of THF was added dropwise and the reaction mixture was stirred for 2.5 hours after which it was quenched with 1 mL of H₂O and brought to room temperature. The product was extracted with EtOAc then the combined organic extracts were washed with brine, dried with Na₂SO₄, filtered, and concentrated via rotary evaporation. The crude product was purified by silica gel chromatography (0-5% EtOAc in hexanes) to afford 84 mg (0.37 mmol, 92% yield) of title compound as a thick, colorless oil. ¹H NMR (400 MHz, C₆D₆) δ7.45-7.38 (m, 2H), 7.05-6.93 (m, 3H), 5.76 (ddd, J=15.4, 11.1, 3.8 Hz, 1H), 5.42 (ddd, J=15.6, 11.2, 3.3 Hz, 1H), 2.55-2.32 (m, 3H), 2.19-2.09 (m, 1H), 2.02-1.79 (m, 3H), 1.73 (app td, J=11.3, 4.6 Hz, 1H), 1.68-1.55 (m, 2H), 1.31 (s, 1H). ¹³C NMR (101 MHz, C₆D₆) δ 135.7 (CH), 131.8 (CH), 131.3 (CH), 128.6 (CH), 128.3 (CH), 123.9 (C), 98.8 (C), 81.9 (C), 68.2 (C), 49.6 (CH₂), 42.1 (CH₂), 34.1 (CH₂), 30.4 (CH₂), 28.1 (CH₂). FTMS (ESI+) calculated [M+H]⁺ for C₁₆H₁₉O 227.1436; found 227.1426.

### Example 7: 5-ax-Hydroxy-5-eq-phenyl-trans-cyclooctene (8)

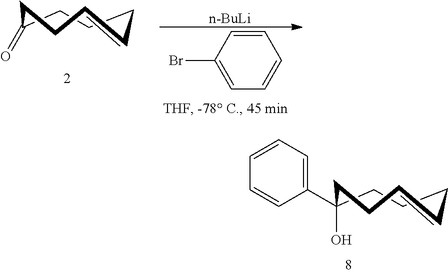

A round bottom flask equipped with a magnetic stir bar was charged with phenyl bromide (84 μL, 0.806 mmol) and 1.6 mL of THF. The flask was then cooled by a bath of dry ice/acetone. n-Butyllithium (350 μL, 2.5 M in hexanes) was added dropwise and the reaction mixture was stirred for 30 minutes. (E)-Cyclooct-4-enone (50 mg, 0.403 mmol) in 100 μL of THF was added dropwise. The reaction was monitored by TLC and quenched after 45 minutes with 1 mL of saturated NH₄Cl aqueous solution. The product was extracted with Et₂O, dried with Na₂SO₄, filtered, and concentrated via rotary evaporation. The crude oil was purified via silica gel chromatography (0-5% EtOAc in hexanes) to afford 80 mg (0.40 mmol, 98% yield) of the title compound as a white solid. ¹H NMR (400 MHz, C₆D₆) δ 7.33 (dd, J=8.6, 1.3 Hz, 2H), 7.25-7.18 (m, 2H), 7.12-7.04 (m, 1H), 5.65-5.45 (m, 2H), 2.38-2.21 (m, 1H), 2.17-2.07 (m, 1H), 2.00-1.88 (m, 2H), 1.88-1.78 (m, 2H), 1.78-1.56 (m, 3H), 1.55-1.43 (m, 1H), 1.29 (s, 1H). ¹³C NMR (101 MHz, C₆D₆) δ 156.0 (C), 134.5 (CH), 132.9 (CH), 128.3 (CH), 125.9 (CH), 123.6 (CH), 74.1 (C), 50.8 (CH₂), 42.2 (CH₂), 34.2 (CH₂), 31.3 (CH₂), 28.3 (CH₂). FTMS (ESI+) calculated [M+H]⁺ for C₁₄H₁₉O 203.1436; found 203.1427.

### Example 8: (E)-Methyl-2-(1-ax-hydroxycyclooct-4-en-1-yl)acetate (9)

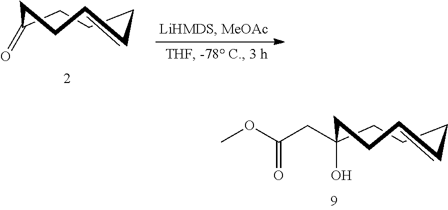

A round bottom flask equipped with a magnetic stir bar was charged with LiHMDS (4.1 mL, 1M in THF) and cooled by a bath of dry ice/acetone. Methyl acetate (0.3 mL, 3.72 mmol) was added dropwise and the reaction mixture was stirred for 30 minutes. (E)-Cyclooct-4-enone (308 mg, 2.48 mmol) in 250 μL of THF was added dropwise over 10 minutes. The reaction mixture was stirred for 3 hours then quenched with 3 mL of saturated aq. NH₄Cl and brought to room temperature. The product was extracted from the aqueous phase with Et₂O, dried with MgSO₄, filtered, and concentrated via rotary evaporation. The crude product was purified by silica gel chromatography using 30% CH₂Cl₂ in hexanes to elute traces of starting material then was changed to 5% Et₂O in hexanes. The purification afforded 423 mg (2.14 mmol, 86% yield) of the title compound as a white solid. ¹H NMR (400 MHz, C₆D₆) δ 5.97 (ddd, J=15.4, 11.3, 3.7 Hz, 1H), 5.44 (ddd, J=15.4, 11.3, 3.3 Hz, 1H), 3.76 (s, 1H), 3.19 (s, 3H), 2.67 (app qd, J=11.8, 4.8 Hz, 1H), 2.29-2.22 (m, 1H), 2.13 (app d, J=15.8 Hz, 2H), 2.04-1.89 (m, 2H), 1.83-1.70 (m, 2H), 1.68-1.59 (m, 1H), 1.49 (dd, J=15.1, 6.6 Hz, 1H), 1.35 (td, J=13.3, 4.8 Hz, 1H), 1.26 (dd, J=15.2, 11.8 Hz, 1H). ¹³C NMR (101 MHz, C₆D₆) δ 173.9 (C), 135.5 (CH), 131.5 (CH), 70.9 (C), 51.1 (CH₃), 50.3 (CH₂), 47.9 (CH₂), 39.7 (CH₂), 34.7 (CH₂), 30.7 (CH₂), 27.6 (CH₂). FTMS (ESI+) calculated [M+H]⁺ for C₁₁H₁₉O₃ 199.1334; found 199.1325.

### Example 9: (E)-2-(1-ax-Hydroxycyclooct-4-en-1-yl)acetonitrile (10)

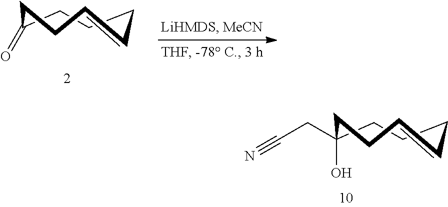

A round bottom flask equipped with a magnetic stir bar was charged with LiHMDS (17.7 mL, 1 M in THF) and THF (7.6 mL) and was then cooled by a bath of dry ice/acetone. Anhydrous acetonitrile (841 μL, 16.1 mmol) was added dropwise and the reaction mixture was stirred for 30 minutes. (E)-Cyclooct-4-enone (200 mg, 1.61 mmol) in THF (2.1 mL) was added dropwise to the flask over 15 minutes. The reaction was monitored by TLC and a change in color from yellow to orange was observed. The reaction mixture was quenched after 2 hours with 5 mL of saturated NH₄Cl aqueous solution then brought to room temperature. The aqueous layer was extracted with Et₂O, dried with MgSO₄, filtered, and concentrated via rotary evaporation. The crude oil was purified by silica gel chromatography (10% EtOAc in hexanes) to afford 261 mg (1.58 mmol, 98% yield) of the title compound as a white solid. ¹H NMR (400 MHz, C₆D₆) δ 5.31 (ddd, J=16.0, 10.6, 3.7 Hz, 1H), 5.20 (ddd, J=15.9, 10.9, 3.2 Hz, 1H), 2.13-1.96 (m, 2H), 1.85-1.75 (m, 1H), 1.63-1.51 (m, 4H), 1.48-1.40 (m, 2H), 1.37-1.21 (m, 2H), 1.10-0.96 (m, 2H). ¹³C NMR (101 MHz, C₆D₆) δ 134.3 (CH), 132.4 (CH), 117.7 (C), 71.2 (C), 46.8 (CH₂), 38.7 (CH₂), 36.8 (CH₂), 33.9 (CH₂), 30.3 (CH₂), 27.4 (CH₂). FTMS (ESI+) calculated [M+H]⁺ for C₁₀H₁₆NO 166.1232; found 166.1224.

### Example 10A: (E)-N-Hydroxysuccinyl 2-(1-ax-hydroxycyclooct-4-en-1-yl)acetate (11)

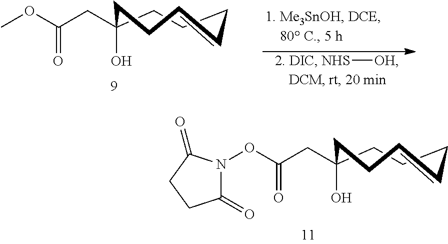

Procedure 1: A round bottom flask equipped with a magnetic stir bar and a condenser was charged with (E)-methyl-2-(1-ax-hydroxycyclooct-4-en-1-yl)acetate (412 mg, 2.08 mmol), Me₃SnOH (3.8 g, 21 mmol), and dichloroethane (21 mL). The reaction flask was immersed in an 80° C. oil bath for 5 hours then was cooled to room temperature. The reaction mixture was directly loaded onto a silica gel column and chromatographed (20-50% EtOAc in hexanes) to afford a carboxylic acid intermediate as a white solid that was used directly in the next step. To the white solid in CH₂Cl₂ (21 mL) was added N-hydroxysuccinimide (359 mg, 3.12 mmol) and N,N′-diisopropylcarbodiimide (0.49 mL, 3.12 mmol). The mixture was stirred at room temperature and monitored by TLC. It was quenched with 8 mL of H₂O after 20 minutes of stirring. The product was extracted from the aqueous phase with CH₂Cl₂, washed with brine, dried with Na₂SO₄, filtered, and concentrated via rotary evaporation. The crude product was purified by silica gel chromatography (0-2% acetone in CH₂Cl₂) to afford 521 mg (1.85 mmol, 89% yield) of the title compound as a white solid.

### Example 10B: (E)-N-Hydroxysuccinyl 2-(1-ax-hydroxycyclooct-4-en-1-yl)acetate (11)

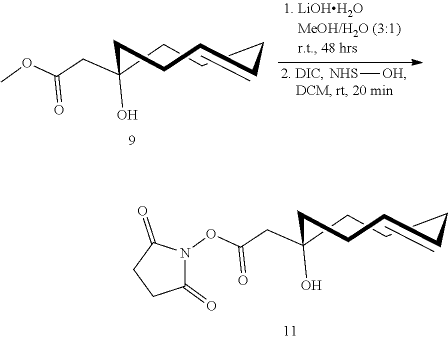

Procedure 2: A 7 mL vial equipped with a magnetic stir bar was charged with (E)-methyl-2-(1-ax-hydroxycyclooct-4-en-1-yl)acetate (100 mg, 0.505 mmol), 2.5 mL of 3:1 MeOH/H₂O, and lithium hydroxide monohydrate (64 mg, 1.52 mmol). The reaction mixture was stirred for 48 hours. The methanol was removed by rotary evaporation and aqueous solution was diluted with 4 mL of ethyl acetate. The mixture was acidified to ˜pH 4 via the dropwise addition of 2M HCl while stirring vigorously. The ethyl acetate was removed, and the aqueous layer was extracted with ethyl acetate several more times. The acidification and extraction processes were repeated until all product was extracted (TLC monitored). The combined organic layers were washed with brine, dried with Na₂SO₄, filtered, and concentrated by rotary evaporation to afford a carboxylic acid intermediate as a white solid that was used directly in the next step. The solid was diluted in 2.9 mL of CH₂Cl₂ and transferred to a 50 mL round bottom flask. N-hydroxysuccinimide (50 mg, 0.432 mmol) was added followed by N,N′-diisopropylcarbodiimide (68 μL, 0.432 mmol). The reaction mixture was stirred at room temperature and monitored by TLC. It was quenched with 2 mL of H₂O after 20 minutes. The aqueous layer was extracted with CH₂Cl₂, the organics were washed with brine then dried with Na₂SO₄, filtered, and concentrated by rotary evaporation. The crude product was purified by silica gel chromatography (0-2% acetone in CH₂Cl₂) to afford 66 mg (0.23 mmol, 46% yield) of the title compound as a white solid.

¹H NMR (400 MHz, C₆D₆) δ 5.75 (ddd, J=15.1, 10.9, 3.7 Hz, 1H), 5.36 (ddd, J=15.4, 11.4, 3.3 Hz, 1H), 2.55 (s, 1H), 2.50 (app qd, J=12.1, 5.0 Hz, 1H), 2.29 (d, J=14.7 Hz, 1H), 2.25 (d, J=14.7 Hz, 1H), 2.20-2.07 (m, 1H), 2.03-1.91 (m, 1H), 1.91-1.78 (m, 2H), 1.81-1.34 (m, 8H), 1.28 (dd, J=15.2, 11.6 Hz, 1H). ¹³C NMR (101 MHz, C₆D₆) δ 168.9 (C), 167.9 (C), 135.2 (CH), 131.6 (CH), 71.8 (C), 48.6 (CH₂), 47.5 (CH₂), 39.0 (CH₂), 34.4 (CH₂), 30.6 (CH₂), 27.6 (CH₂), 25.2 (CH₂). FTMS (ESI+) calculated [M+H]⁺ for C₁₄H₂₀NO₅ 282.1341; found 282.1340.

### Example 11: (R,E)-2-(6-(dimethylamino)-3-(dimethyliminio)-3H-xanthen-9-yl)-5-((5-(2-(1-hydroxycyclooct-4-en-1-yl)acetamido)pentyl)carbamoyl)benzoate (TAMRA-a-TCO) (12)

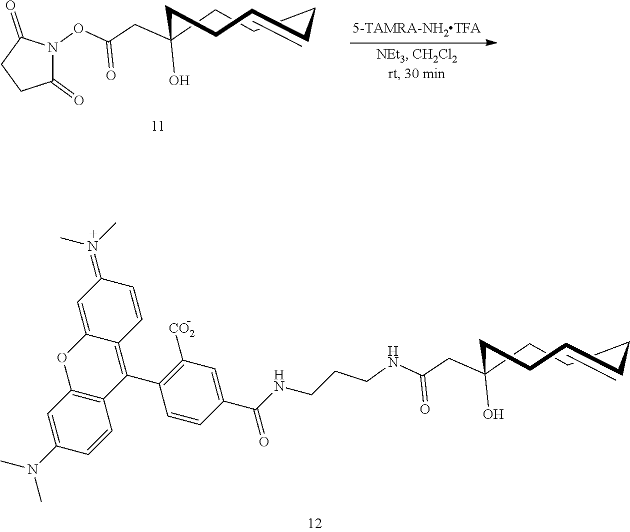

To a solution of 5-TAMRA Cadaverine TFA salt (5.1 mg, 8.1 μmol), CH₂Cl₂ (4 mL), and triethylamine (5.6 μL, 40 μmol) in a 25 mL vial was added (E)-N-hydroxysuccinyl-2-(1-ax-hydroxycyclooct-4-en-1-yl)acetate (5.3 mg, 19 μmol) in 1.5 mL of CH₂Cl₂. The reaction mixture was stirred for 30 minutes at room temperature before concentrating by rotary evaporation and redissolving in a minimal amount of 1:1 MeOH/H₂O. The solution was directly injected onto a revered phase prepackaged C-18 column (14 g) and the separation was conducted on the Teledyne Isco (Combiflash® RF) using a 0-100% MeOH in H₂O gradient. The fractions containing product were concentrated using the Biotage® V-10 Touch evaporation system to afford 4.3 mg (6.3 μmol, 79% yield) the title product as a purple solid. ¹H NMR (600 MHz, MeOD) δ 8.51 (d, J=1.8 Hz, 1H), 8.04 (dd, J=7.9, 1.9 Hz, 1H), 7.36 (d, J=7.9 Hz, 1H), 7.26 (d, J=9.5 Hz, 2H), 7.02 (dd, J=9.4, 2.5 Hz, 2H), 6.93 (d, J=2.5 Hz, 2H), 5.64 (ddd, J=14.6, 10.5, 3.6 Hz, 1H), 5.51 (ddd, J=14.7, 11.1, 2.9 Hz, 1H), 3.46 (t, J=7.0 Hz, 2H), 3.28 (s, 12H), 3.22 (t, J=6.9 Hz, 2H), 2.43 (qd, J=12.0, 4.6 Hz, 1H), 2.26-2.16 (m, 3H), 2.04-1.99 (m, 1H), 1.94-1.88 (m, 1H), 1.85-1.75 (m, 2H), 1.74-1.65 (m, 5H), 1.63-1.55 (m, 2H), 1.51-1.43 (m, 3H). FTMS (ESI+) calculated [M+H]⁺ for C₄₀H₄₉N₄O₆ 681.3652; found 681.3644.

### Example 12: (E)-2-(1-ax-Hydroxycyclooct-4-en-1-yl)acetohydrazide (13)

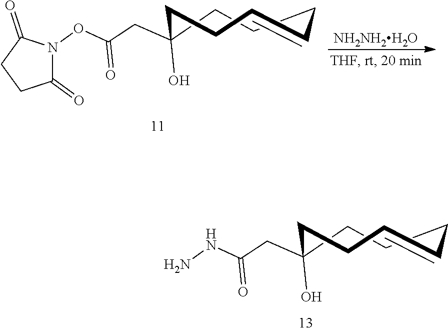

A round bottom flask equipped with a magnetic stir bar was charged with (E)-N-hydroxysuccinyl-2-(1-ax-hydroxycyclooct-4-en-1-yl)acetate (50 mg, 0.18 mmol), THF (1.8 mL), and hydrazine monohydrate (17.4 μL, 0.355 mmol). The reaction was monitored by TLC and concentrated after 20 minutes of stirring. The crude mixture was purified by silica gel chromatography (0-2.5% MeOH in CH₂Cl₂) to afford 35 mg (0.18 mmol, quant.) of the title compound as a white solid. The compound was stored at −20° C. as a solution in Et₂O. ¹H NMR (400 MHz, DMSO) δ 5.56 (ddd, J=15.6, 10.2, 3.2 Hz, 1H), 5.44 (ddd, J=15.9, 10.9, 3.2 Hz, 1H), 3.52 (br s, 4H), 2.32 (app qd, J=11.5, 4.6 Hz, 1H), 2.20-2.09 (m, 1H), 2.06 (app d, J=2.3 Hz, 2H), 1.98-1.88 (m, 1H), 1.86-1.76 (m, 1H), 1.76-1.65 (m, 2H), 1.65-1.51 (m, 3H), 1.39-1.26 (m, 1H). ¹³C NMR (101 MHz, DMSO) δ 171.6 (C), 134.9 (CH), 131.7 (CH), 71.0 (C), 49.4 (CH₂), 47.6 (CH₂), 39.0 (CH₂), 34.2 (CH₂), 30.2 (CH₂), 27.2 (CH₂). FTMS (ESI+) calculated [M+H]⁺ for C₁₀H₁₉N₂O₂ 199.1446; found 199.1436.

### Example 13: 5-ax-Hydroxy-5-eq-(2-hydroxyethyl)-trans-cyclooctene (14)

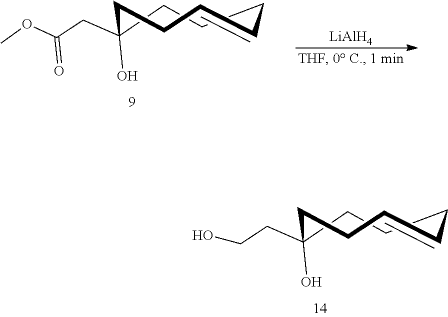

A Schlenk flask equipped with a magnetic stir bar was charged with LiAlH₄ (57.3 mg, 1.51 mmol) and THF (2.2 mL) then chilled to 0° C. A solution of (E)-methyl-2-(1-ax-hydroxycyclooct-4-en-1-yl)acetate (100 mg, 0.505 mmol) in THF (0.55 mL) was added to the flask dropwise and some bubbling was observed. After addition, the mixture was immediately diluted with 6 mL of THF then quenched by the dropwise addition of 1.2 mL of water and 1.2 mL of 10% NaOH in water. The mixture was stirred for 5 minutes then allowed to warm to room temperature, after which Na₂SO₄ was added. The reaction mixture was stirred another 5 minutes before filtering, rinsing the solids with Et₂O, and concentrating via rotary evaporation. The product was purified by silica gel chromatography (20-40% Et₂O in hexanes) to afford 76 mg (0.45 mmol, 89% yield) of the title compound as a white solid. ¹H NMR (600 MHz, C₆D₆) δ 5.57 (ddd, J=15.7, 10.7, 3.8 Hz, 1H), 5.47 (ddd, J=15.8, 11.2, 3.3 Hz, 1H), 3.63-3.47 (m, 2H), 2.41 (qd, J=12.0, 4.9 Hz, 1H), 2.23 (s, 1H), 2.21-2.14 (m, 1H), 2.06-2.01 (m, 1H), 2.01-1.95 (m, 1H), 1.88-1.81 (m, 1H), 1.79-1.69 (m, 2H), 1.66-1.54 (m, 2H), 1.43 (ddd, J=14.3, 7.3, 4.4 Hz, 1H), 1.33-1.24 (m, 2H), 1.13-1.07 (m, 1H). ¹³C NMR (101 MHz, C₆D₆) δ 133.9 (CH), 133.4 (CH), 73.5 (C), 60.0 (CH₂), 48.13 (CH₂), 48.08 (CH₂), 39.3 (CH₂), 34.5 (CH₂), 30.7 (CH₂), 27.5 (CH₂). FTMS (ESI+) calculated [M+H]⁺ for C₁₀H₁₉O₂ 171.1385; found 171.1376.

### Example 14: 5-ax-Hydroxy-5-eq-(2-(prop-2-yn-1-yloxy)ethyl)-trans-cyclooctene (15)

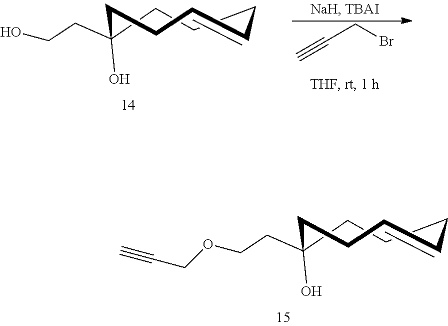

A round bottom flask equipped with a magnetic stir bar was charged with 5-ax-hydroxy-5-eq-(2-hydroxyethyl)-trans-cyclooctene (30 mg, 0.18 mmol) and THF (0.9 mL). NaH (14 mg, 0.35 mmol, 60% in mineral oil) was added in one portion and the reaction mixture was stirred for 30 minutes. Propargyl bromide (38 μL, 0.35 mmol, 9.2 mol/L in toluene) was added dropwise followed by the addition of tetrabutylammonium iodide (2.8 mg, 8.8 μmol). The reaction was monitored by TLC and quenched with 1 mL of water after stirring 1 hour. The product was extracted from the aqueous phase with Et₂O, washed with brine, dried with Na₂SO₄, filtered, and concentrated by rotary evaporation. The product was purified by silica gel chromatography (0-10% Et₂O in hexanes) to afford 35.1 mg (0.167 mmol, 96% yield) of the title compound as a pale-yellow oil. ¹H NMR (600 MHz, C₆D₆) δ 5.82 (ddd, J=15.3, 11.1, 3.8 Hz, 1H), 5.51 (ddd, J=15.4, 11.4, 3.4 Hz, 1H), 3.68 (dd, J=15.9, 2.3 Hz, 1H), 3.67 (dd, J=15.9, 2.3 Hz, 1H), 3.47-3.37 (m, 2H), 2.57 (app qd, J=11.9, 4.8 Hz, 1H), 2.29-2.24 (m, 1H), 2.12 (s, 1H), 2.07-2.01 (m, 1H), 1.98 (t, J=2.4 Hz, 1H), 1.94-1.75 (m, 3H), 1.68-1.58 (m, 2H), 1.55 (ddd, J=14.3, 6.8, 5.7 Hz, 1H), 1.46 (app dt, J=14.2, 6.0 Hz, 1H), 1.36 (app td, J=13.1, 4.7 Hz, 1H), 1.18 (dd, J=14.4, 11.6 Hz, 1H). ¹³C NMR (151 MHz, C₆D₆) δ 134.7 (CH), 132.6 (CH), 79.9 (C), 74.7 (CH), 72.1 (C), 67.2 (CH₂), 58.2 (CH₂), 48.2 (CH₂), 46.8 (CH₂), 39.4 (CH₂), 34.8 (CH₂), 30.8 (CH₂), 27.6 (CH₂). FTMS (ESI+) calculated [M+H]⁺ for C₁₃H₂₁O₂ 209.1541; found 209.1536.

### Example 15: 5-ax-Hydroxy-5-eq-(2-aminoethyl)-trans-cyclooctene (16)

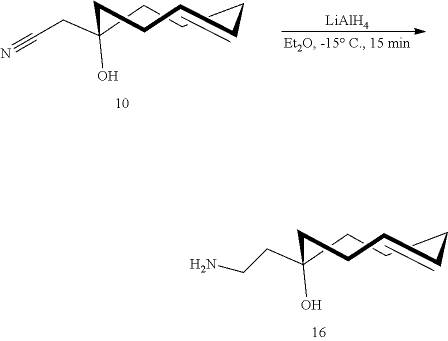

A round bottom flask equipped with a magnetic stir bar was charged with (E)-2-(1-ax-hydroxycyclooct-4-en-1-yl)acetonitrile (50 mg, 0.30 mmol) and Et₂O (3.4 mL) then was chilled to −15° C. LiAlH₄ (354 mg, 0.91 mmol) was added in portions and the reaction was monitored by TLC. The mixture was quenched after 15 minutes of stirring with a 1 drop of water followed by 2 drops of 10% aq. NaOH and 0.1 mL of water. The reaction mixture was warmed to room temperature and stirred for 10 minutes. MgSO₄ was added and the mixture was stirred another 15 minutes. The solids were filtered off and rinsed with ethyl acetate. The product was concentrated via rotary evaporation to afford 48 mg (0.28 mmol, 93% yield) of a colorless oil. ¹H NMR (400 MHz, C₆D₆) δ 6.08 (ddd, J=15.5, 11.0, 3.8 Hz, 1H), 5.61 (ddd, J=15.5, 11.6, 3.6 Hz, 1H), 2.84 (app qd, J=11.6, 4.2 Hz, 1H), 2.49-2.32 (m, 3H), 2.25-2.01 (m, 2H), 2.00-1.83 (m, 2H), 1.79-1.69 (m, 1H), 1.63 (dd, J=15.0, 6.9 Hz, 1H), 1.41 (app td, J=13.1, 4.7 Hz, 1H), 1.33-1.16 (m, 2H), 1.15-1.04 (m, 1H). ¹³C NMR (101 MHz, C₆D₆) δ 135.1 (CH), 132.4 (CH), 72.9 (C), 49.3 (CH₂), 47.1 (CH₂), 39.9 (CH₂), 38.4 (CH₂), 35.2 (CH₂), 31.0 (CH₂), 27.5 (CH₂). FTMS (ESI+) calculated [M+H]⁺ for C₁₀H₂₀NO 170.1544; found 170.1539.

### Example 16: (R,E)-N-(2-(2,5-dioxo-2,5-dihydro-1H-pyrrol-1-yl)ethyl)-2-(1-hydroxycyclooct-4-en-1-yl)acetamide (17)

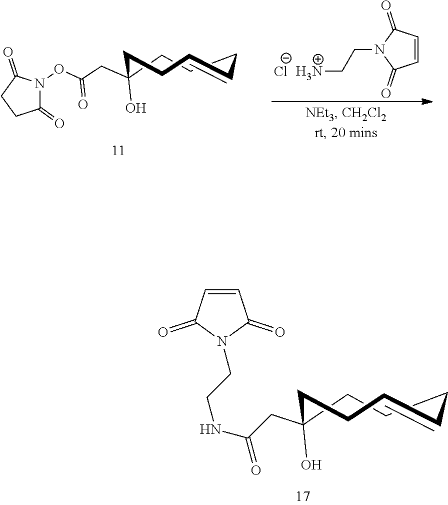

A round bottomed flask equipped with a magnetic stir bar was charged with 1-(2-aminoethyl)maleimide hydrochloride (103 mg, 0.586 mmol), triethylamine (0.27 mL, 1.955 mmol), and CH₂Cl₂ (3.9 mL) and was stirred briefly before (E)-N-hydroxysuccinyl 2-(1-ax-hydroxycyclooct-4-en-1-yl)acetate (110 mg, 0.391 mmol) in CH₂Cl₂ (3.9 mL) was added. The reaction was monitored by TLC. After 20 minutes, the reaction was loaded directly onto a silica gel column and chromatographed (30-60% ethyl acetate in hexanes) to afford 103 mg (0.336 mmol, 86% yield) of the title compound as a white solid. The compound was stored at −20° C. as a solution in methanol. ¹H NMR (400 MHz, C₆D₆) δ 6.06 (ddd, J=15.3, 11.2, 3.7 Hz, 1H), 5.72 (s, 2H), 5.54 (ddd, J=15.4, 11.4, 3.3 Hz, 1H), 4.94 (s, 1H), 4.76 (brs, 1H), 3.20-3.05 (m, 2H), 3.03-2.88 (m, 2H), 2.77 (app qd, J=11.8, 4.6 Hz, 1H), 2.37-2.26 (m, 1H), 2.15-1.96 (m, 2H), 1.93-1.75 (m, 3H), 1.73-1.63 (m, 2H), 1.48 (dd, J=14.9, 6.6 Hz, 1H), 1.36 (dd, J=13.0, 4.7 Hz, 1H), 1.32-1.23 (m, 1H). ¹³C NMR (101 MHz, C₆D₆) δ 173.3 (C), 170.5 (C), 135.7 (CH), 133.6 (CH), 131.7 (CH), 71.4 (C), 51.0 (CH₂), 48.3 (CH₂), 39.9 (CH₂), 38.1 (CH₂), 37.4 (CH₂), 34.9 (CH₂), 30.7 (CH₂), 27.8 (CH₂). FTMS (ESI+) calculated [M+H]⁺ for C₁₆H₂₃N₂O₄ 307.1658; found 307.1642.

### Example 17: (E)-Cyclooct-4-enone Oxime (18)

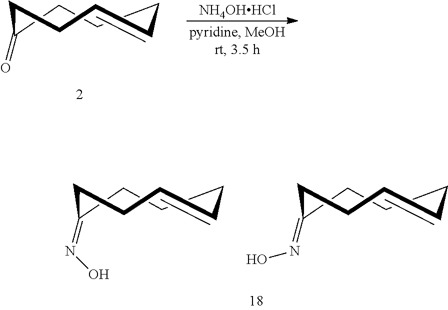

An oven-dried, 7 mL vial equipped with a magnetic stir bar was charged with hydroxylamine hydrochloride (196 mg, 2.82 mmol), 0.26 mL of pyridine, and 1 mL of MeOH and was then stirred for 10 minutes. (E)-Cyclooct-4-enone (100 mg, 0.805 mmol) in 1 mL of MeOH was added, and the reaction was monitored by TLC. After 3.5 hours of stirring, the mixture was partitioned between Et₂O and water. The product was extracted from the aqueous phase with Et₂O, dried with Na₂SO₄, filtered, then concentrated by rotary evaporation. The crude product was purified by silica column chromatography (10-20% EtOAc in hexanes) to afford 91 mg (0.65 mmol, 81% yield) of the title compound as a white solid and as a 4:1 mixture of geometric isomers as judged by ¹H NMR.

Peaks attributed to major product: ¹H NMR (400 MHz, CDCl₃) δ 8.05 (br s, 1H), 5.77-5.62 (m, 1H), 5.48-5.34 (m, 1H), 2.82 (ddd, J=13.1, 5.6, 1.7 Hz, 1H), 2.72-2.59 (m, 1H), 2.56-2.15 (m, 5H), 2.08-1.85 (m, 2H), 1.41 (td, J=12.8, 1.7 Hz, 1H). ¹³C NMR (101 MHz, CDCl₃) δ 164.0 (C), 135.0 (CH), 132.4 (CH), 42.3 (CH₂), 35.5 (CH₂), 34.0 (CH₂), 32.5 (CH₂), 28.2 (CH₂).

Peaks attributed to minor product: ¹H NMR (400 MHz, CDCl₃) δ 8.05 (br s, 1H), 5.77-5.62 (m, 1H), 5.48-5.34 (m, 1H), 3.41 (app dd, J=11.6, 4.6 Hz, 1H), 2.56-2.15 (m, 5H), 2.08-1.85 (m, 2H), 1.83-1.68 (m, 1H), 1.59 (ddd, J=14.4, 12.8, 1.7 Hz, 1H). ¹³C NMR (101 MHz, CDCl₃) δ 164.7 (C), 134.3 (CH), 133.1 (CH), 37.1 (CH₂), 35.1 (CH₂), 34.1 (CH₂), 33.4 (CH₂), 32.1 (CH₂).

FTMS (ESI+) calculated [M+H]⁺ for C₈H₁₄NO 140.1075; found 140.1070.

### Example 18: (E)-Cyclooct-4-enone O-benzyl Oxime (19)

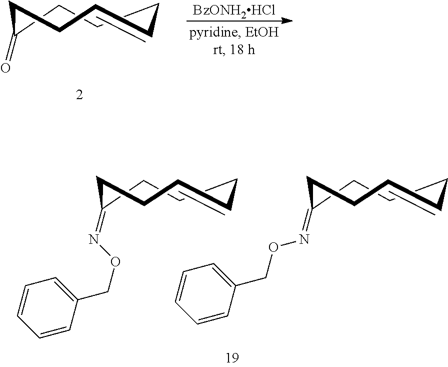

(E)-Cyclooct-4-enone (50 mg, 0.40 mmol), 2.2 mL of ethanol, and 0.2 mL of pyridine were added to a round bottom flask equipped with a magnetic stir bar. O-Benzylhydroxylamine hydrochloride (129 mg, 0.806 mmol) was added in portions while vigorously stirring. The reaction was monitored by TLC and stirred for 18 hours, after which the mixture was concentrated by rotary evaporation and dissolved in a 1:1: CH₂Cl₂ and EtOAc solution. The precipitate formed was filtered off and rinsed with CH₂Cl₂ and EtOAc. The crude product was purified by silica gel chromatography (0-3% Et₂O in hexanes) to afford 76 mg (0.33 mmol, 83% yield) of a white solid that was a 3:1 mixture of geometric isomers as judged by ¹H NMR.

Peaks attributed to major product: ¹H NMR (400 MHz, C₆D₆) δ 7.39-7.30 (m, 2H), 7.21-7.13 (m, 2H), 7.11-7.05 (m, 1H), 5.45-5.28 (m, 2H), 5.22-5.05 (m, 2H), 2.73 (ddd, J=28.1, 12.8, 5.3 Hz, 2H), 2.41-2.24 (m, 2H), 2.23-2.03 (m, 2H), 1.97-1.81 (m, 1H), 1.78-1.60 (m, 2H), 1.03 (app td, J=12.8, 1.7 Hz, 1H). ¹³C NMR (101 MHz, C₆D₆) δ 162.8 (C), 139.3 (C), 134.8 (CH), 132.6 (CH), 128.6 (CH), 75.8 (CH₂), 42.3 (CH₂), 35.9 (CH₂), 34.1 (CH₂), 32.9 (CH₂), 28.9 (CH₂).

Peaks attributed to minor product: ¹H NMR (400 MHz, C₆D₆) δ 7.39-7.30 (m, 2H), 7.21-7.13 (m, 2H), 7.11-7.05 (m, 1H), 5.45-5.28 (m, 2H), 5.22-5.05 (m, 2H), 3.40 (app dd, J=11.8, 4.7 Hz, 1H), 2.61-2.46 (m, 1H), 2.41-2.24 (m, 1H), 2.23-2.03 (m, 2H), 1.97-1.81 (m, 1H), 1.78-1.60 (m, 2H), 1.51 (app td, J=12.2, 5.1 Hz, 1H), 1.13 (ddd, J=14.3, 12.6, 1.8 Hz, 1H). ¹³C NMR (101 MHz, C₆D₆) δ 163.5 (C), 139.5 (C), 134.4 (CH), 133.0 (CH), 128.5 (CH), 75.7 (CH₂), 37.7 (CH₂), 35.1 (CH₂), 34.3 (CH₂), 33.8 (CH₂), 32.37 (CH₂).

FTMS (ESI+) calculated [M+H]⁺ for C₁₅H₂₀NO 230.1545; found 230.1535.

### OXO-TCO-Tamra Synthesis

(R,E)-(3,4,7,8-tetrahydro-2H-oxocin-2-yl)methanol (oxo-TCO) was synthesized according to a previously published procedure (Brown et al., Chem. Soc. Rev. 2017, 46, 6532-6552).

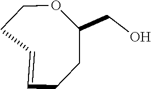

### Example 19: (R,E)-4-nitrophenyl ((3,4,7,8-tetrahydro-2H-oxocin-2-yl)methyl) carbonate

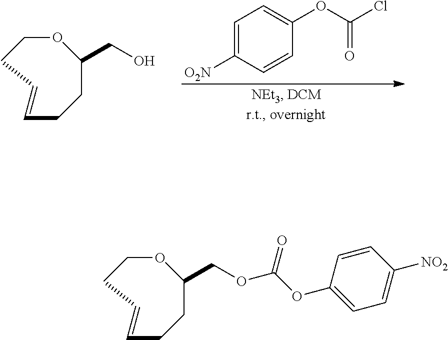

A dry round bottom flask was charged with (R,E)-(3,4,7,8-tetrahydro-2H-oxocin-2-yl)methanol (202 mg, 1.42 mmol) and a magnetic stir bar. 4-Nitrophenyl chloroformate (344 mg, 1.70 mmol) dissolved in anhydrous CH₂Cl₂ (7 mL) and pyridine (0.458 mL, 5.68 mmol) was added by syringe. After stirring under nitrogen at room temperature overnight, the resulting solution was concentrated by rotary evaporation. A yellow oil (400 mg, 1.30 mmol, 92%) was obtained after column chromatography (0-5% EtOAc in CH₂Cl₂). ¹H NMR (400 MHz, C₆D₆) δ 7.73-7.59 (m, 2H), 6.83-6.66 (m, 2H), 5.73 (ddd, J=15.4, 11.4, 3.4 Hz, 1H), 5.13 (ddd, J=15.4, 10.9, 3.8 Hz, 1H), 4.03 (dd, J=10.8, 7.6 Hz, 1H), 3.98-3.88 (m, 1H), 3.82 (dd, J=10.8, 4.4 Hz, 1H), 2.91 (td, J=11.7, 3.3 Hz, 1H), 2.86-2.72 (m, 1H), 2.37-2.13 (m, 2H), 1.99-1.76 (m, 2H), 1.70-1.49 (m, 1H), 1.39-1.33 (m, 1H). ¹³C NMR (101 MHz, C₆D₆) δ 155.4 (C), 152.9 (C), 145.6 (C), 140.8 (CH), 127.1 (CH), 125.2 (CH), 121.5 (CH), 82.2 (CH), 74.19 (CH₂), 72.15 (CH₂), 38.2 (CH₂), 37.2 (CH₂), 34.1 (CH₂). HRMS (ESI+) calculated [M+H]⁺ for C₁₅H₁₈O₆N 308.1134; found 308.1128.

### Example 20: (S,E)-2-(6-(dimethylamino)-3-(dimethyliminio)-3H-xanthen-9-yl)-5-((5-((((3,4,7,8-tetrahydro-2H-oxocin-2-yl)methoxy)carbonyl)amino)pentyl)carbamoyl)benzoate (TAMRA-oxo-TCO)

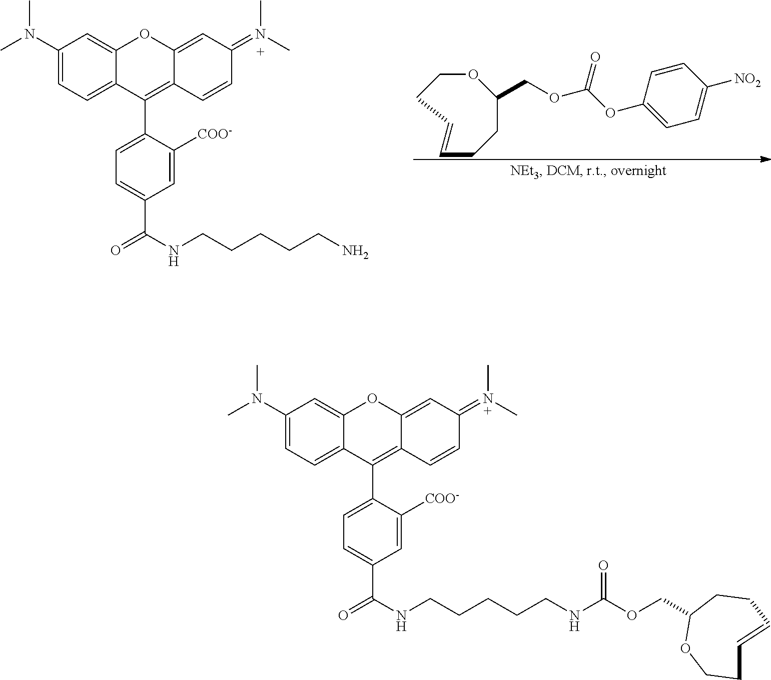

A dry round bottom flask was charged with 5-TAMRA Cadaverine TFA salt (3.8 mg, 6.1 μmol) and a magnetic stir bar. Anhydrous CH₂Cl₂ (1 mL), triethylamine (8.5 mL, 61 μmol) and (R,E)-4-nitrophenyl ((3,4,7,8-tetrahydro-2H-oxocin-2-yl)methyl) carbonate (3.7 mg, 12 μmol) were added by syringe. After stirring under nitrogen at room temperature overnight, the resulting mixture was concentrated by rotary evaporation. A purple solid (1.6 mg, 2.3 μmol, 39%) was obtained after reverse phase column chromatography with a 14 g Yamazan column (0-50% acetonitrile in H₂O with 0.05% NH₄OH). ¹H NMR (600 MHz, MeOD) δ 8.50 (d, J=1.8 Hz, 1H), 8.04 (dd, J=7.9, 1.9 Hz, 1H), 7.35 (d, J=7.9 Hz, 1H), 7.25 (d, J=9.5 Hz, 2H), 7.02 (dd, J=9.5, 2.5 Hz, 2H), 6.93 (d, J=2.4 Hz, 2H), 5.67 (ddd, J=15.4, 11.4, 3.4 Hz, 1H), 5.40 (ddd, J=15.5, 10.8, 3.9 Hz, 1H), 4.02 (dd, J=12.0, 6.1 Hz, 1H), 3.92 (dd, J=10.9, 7.5 Hz, 1H), 3.85 (dd, J=11.1, 4.8 Hz, 1H), 3.46 (t, J=7.1 Hz, 2H), 3.28 (s, 12H), 3.13 (t, J=6.9 Hz, 2H), 2.46-2.41 (m, 1H), 2.35-2.26 (m, 1H), 2.25-2.13 (m, 2H), 1.87 (dd, J=14.2, 5.0 Hz, 1H), 1.74-1.65 (m, 3H), 1.62-1.54 (m, 3H), 1.54-1.42 (m, 3H). HRMS (ESI+) calculated for [M+H]⁺ C₃₉H₄₇O₇N₄ 683.3445; found 683.3428.

### Stability Assays

### Example 21: Stability of TCO 14 in methanol-d₄ (35 mM)

A solution of 14 (4.5 mg, 26 μmol) in methanol-d₄ (750 μL) was monitored by quantitative ¹H NMR on a 600 MHz instrument to observe the decomposition of the trans-isomer over time. Methyl tert-butyl ether (3.1 μL, 26 μmol) was used as an internal standard. There was no observable decomposition of TCO 14 after 7 days (FIG. 6A). The results were plotted using Prism software (Version 8.00, GraphPad Software Inc). A waterfall plot of the ¹H NMR spectra is provided.

### Example 22: Stability of TCO 4a in methanol-d₄ (35 mM)

A solution of 4a (3.3 mg, 26 μmol) in methanol-d₄ (750 μL) was monitored by quantitative ¹H NMR on a 600 MHz instrument to observe the decomposition of the trans-isomer over time. Methyl tert-butyl ether (3.1 μL, 26 μmol) was used as an internal standard. There was no observable decomposition of TCO 4a after 7 days (FIG. 6A). The results were plotted using Prism software (Version 8.00, GraphPad Software Inc). A waterfall plot of the ¹H NMR spectra is provided.

### Example 23: Stability of a-TCO 14 in Phosphate Buffered D₂₀, pD=7.4 (33 mM)

A solution of 14 (4.3 mg, 25 μmol) in phosphate buffered D₂O (750 μL, pD=7.4) was monitored by quantitative ¹H NMR on a 600 MHz instrument to observe the decomposition of the trans-isomer over time. Methyl tert-butyl ether (3.0 μL, 26 μmol) was used as an internal standard. After 1 day, 90% of 14 remained and after 5 days, 39% remained (FIG. 6B). The results were plotted using Prism software (Version 8.00, GraphPad Software Inc). A waterfall plot of the ¹H NMR spectra is provided.

### Example 24: Stability of TCO 4a in Phosphate Buffered D₂₀, pD=7.4 (33 mM)

A solution of 4a (3.2 mg, 25 μmol) in phosphate buffered D₂O (750 μL, pD=7.4) was monitored by quantitative ¹H NMR on a 600 MHz instrument to observe the decomposition of the trans-isomer over time. Methyl tert-butyl ether (3.0 μL, 26 μmol) was used as an internal standard. After 1 day, 85% of 4a remained and after 5 days, 35% remained (FIG. 6B). The results were plotted using Prism software (Version 8.00, GraphPad Software Inc). A waterfall plot of the ¹H NMR spectra is provided.

### Example 25: Stability of a-TCO 14 in Phosphate Buffered D₂₀ and Mercaptoethanol, pD=7.4 (25 mM)

TCO 14 (3.1 mg, 18 μmol) in a solution of mercaptoethanol in phosphate buffered D₂O (720 μL, 18 μmol, 25 mM, pD=7.4) was monitored by quantitative 1H NMR on a 600 MHz instrument to observe the decomposition of the trans-isomer over time. Methyl tert-butyl ether (2.1 μL, 18 μmol) was used as an internal standard. After 4 hours, 93% of 14 remained and 49% of 14 remained after 20 hours. (FIG. 6C). The results were plotted using Prism software (Version 8.00, GraphPad Software Inc). A waterfall plot of the ¹H NMR spectra is provided.

### Example 26: Stability of TCO 4a in Phosphate Buffered D₂₀ and Mercaptoethanol, pD=7.4 (25 mM)

TCO 4a (2.3 mg, 18 μmol) in a solution of mercaptoethanol in phosphate buffered D₂O (720 μL, 18 μmol, 25 mM, pD=7.4) was monitored by quantitative 1H NMR on a 600 MHz instrument to observe the decomposition of the trans-isomer over time. Methyl tert-butyl ether (2.1 μL, 18 μmol) was used as an internal standard. After 4 hours, 89% of 4a and 49% of 4a remained after 20 hours. (FIG. 6C). The results were plotted using Prism software (Version 8.00, GraphPad Software Inc). A waterfall plot of the ¹H NMR spectra is provided.

### Stopped-Flow Kinetic Measurements

### Example 27: Kinetic Measurements for the Reaction of 5-ax-hydroxy-5-eq-(2-hydroxyethyl)-trans-cyclooctene (14) and 3,6-dipyridyl-s-tetrazine-mono-succinamic Acid (20)

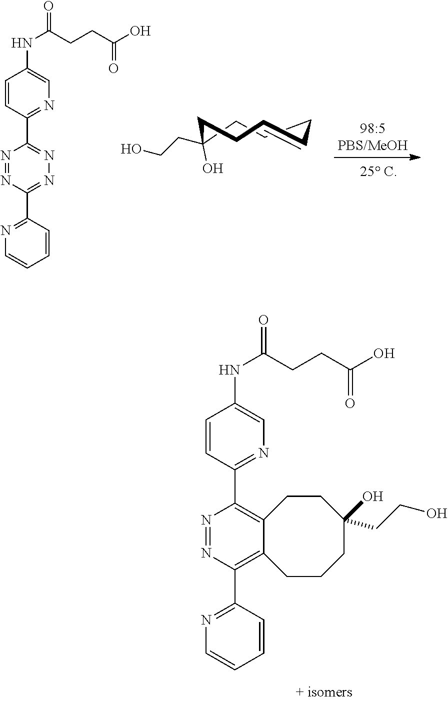

The reaction between 5-ax-hydroxy-5-eq-(2-hydroxyethyl)-trans-cyclooctene 14 and 3,6-dipyridyl-s-tetrazine-mono-succinamic acid 20 was monitored by a SX18MV-R stopped-flow spectrophotometer (Applied Photophysics Ltd.) at 325 nm by measuring the exponential decay of tetrazine under pseudo-first order conditions. Solutions of 14 (0.98, 1.46, 1.95, and 2.63 mM in 9:1 PBS/MeOH) and tetrazine (0.1 mM in PBS) were mixed in the stopped-flow spectrophotometer in equal volumes resulting in final concentrations of 0.49 mM, 0.73 mM, 0.97 mM, and 1.31 mM of 14 and 0.05 mM of tetrazine in 95:5 PBS/MeOH. Three independent samples were prepared for each TCO concentration and measurements were taken in duplicate every 0.1 ms for 0.1 sat 25° C. The observed rates k_{obs} for each run were determined by nonlinear regression analysis of the data points using Prism software (Version 8.00, GraphPad Software Inc). The average k_{obs} values were plotted against the concentrations of 14 to obtain the bimolecular rate constant k₂ from the slope of the plot. The average k₂ was measured as 150,000±8000 M⁻¹s⁻¹.

The observed rates (k_{obs}) of 5-ax-hydroxy-5-eq-(2-hydroxyethyl)-trans-cyclooctene 14 (10-30 equivalents) and 3,6-dipyridyl-s-tetrazine-mono-succinamic acid 20 were measured using stopped-flow kinetics. The final concentrations of 14 after injection were (A) 0.49 mM, (B) 0.73 mM, (C) 0.97 mM, and (D) 1.31 mM and the concentration of tetrazine was 0.05 mM in 95:5 PBS/MeOH at 25° C. Duplicate measurements were obtained for three independent samples for each concentration of 14. The averages and the nonlinear best fit curve) were calculated using Prism software and are summarized below in Table 1.

TABLE 1

| | A (0.49 mM) | B (0.73 mM) | C (0.97 mM) | D (1.31 mM) |
|---|---|---|---|---|
| K_{obs} | 76.13 | 121.4 | 162.4 | 200.2 |
| R² | 0.9995 | 0.9996 | 0.9986 | 0.9934 |

### Example 28: Kinetic Measurements for the Reaction of 5-ax-hydroxy-trans-cyclooctene (4a) and 3,6-dipyridyl-s-tetrazine-mono-succinamic Acid (20)

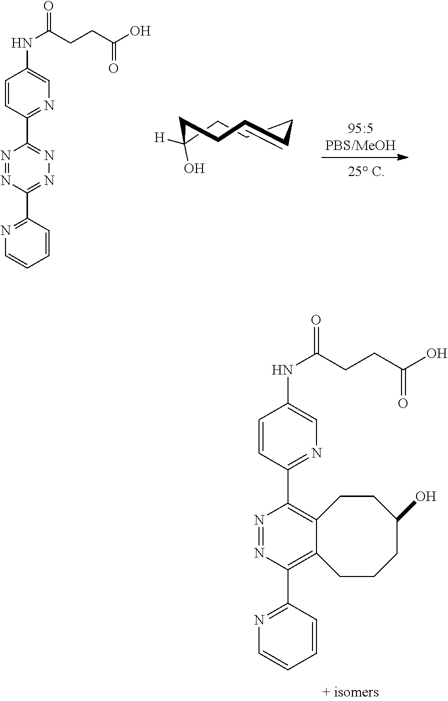

The reaction between 5-ax-hydroxy-trans-cyclooctene 4a and 3,6-dipyridyl-s-tetrazine-mono-succinamic acid 20 was monitored by a SX18MV-R stopped-flow spectrophotometer (Applied Photophysics Ltd.) at 325 nm by measuring the exponential decay of tetrazine under pseudo-first order conditions. Solutions of 4a (100, 200, 300, and 400 μM in 9:1 PBS/MeOH) and tetrazine (20 μM in PBS) were mixed in the stopped-flow spectrophotometer in equal volumes resulting in final concentrations of 50 μM, 100 μM, 150 μM, and 200 μM of 4a and 10 μM of tetrazine in 95:5 PBS/MeOH. Three independent samples were prepared for each TCO concentration and measurements were taken in duplicate every 0.1 ms for 0.2 s at 25° C. The observed rates k_{obs} for each run were determined by nonlinear regression analysis of the data points using Prism software (Version 8.00, GraphPad Software Inc). The average k_{obs} values were plotted against the concentrations of 4a to obtain the bimolecular rate constant k₂ from the slope of the plot. The average k₂ was measured as 70,000±1800 M⁻¹s⁻¹.

The observed rates (k_{obs}) of 5-ax-hydroxy-trans-cyclooctene 4a (5-20 equivalents) and 3,6-dipyridyl-s-tetrazine-mono-succinamic acid 20 were measured using stopped-flow kinetics. The final concentrations of 4a after injection were (A) 50 μM, (B) 100 μM, (C) 150 μM, and (D) 200 μM and the concentration of tetrazine was 10 μM in 95:5 PBS/MeOH at 25° C. Duplicate measurements were obtained for three independent samples for each concentration of 4a. The averages and the nonlinear best fit curve were calculated using Prism software, and summarized below in Table 2.

TABLE 2

| | A (0.49 mM) | B (0.73 mM) | C (0.97 mM) | D (1.31 mM) |
|---|---|---|---|---|
| K_{obs} | 3.407 | 6.966 | 10.09 | 13.86 |
| R² | 0.9983 | 0.9931 | 0.9926 | 0.9943 |

### TAMRA-A-TCO Permeability Assay

### Example 29: 4-((4-(6-methyl-1,2,4,5-tetrazin-3-yl)benzyl)-N-(2-(2-((6-chlorohexyl)oxy)ethoxy)ethyl)amide (21) was Prepared According to a Previously Published Procedure as Described in Scinto et al., J. Am. Chem. Soc. 2019, 141, 10932-10937

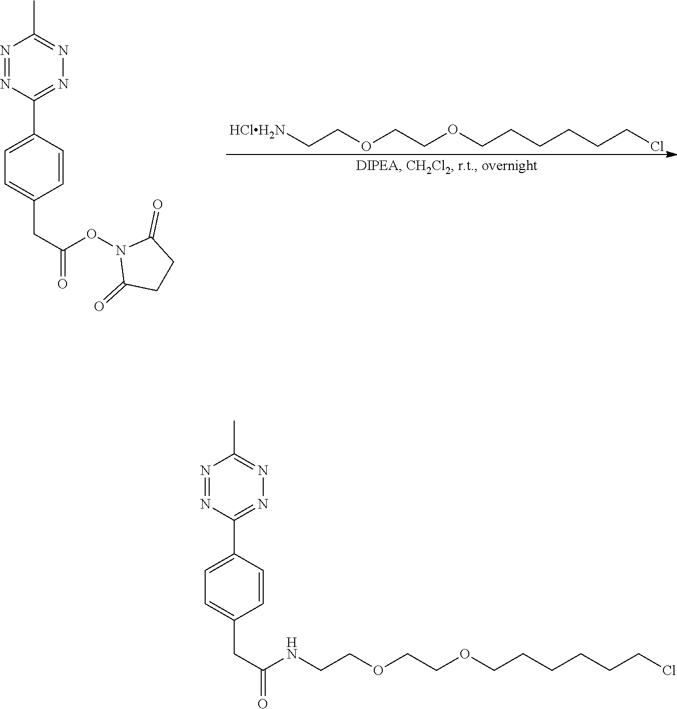

Plasmids: Halo-H2B-GFP and Halo-GAP43-GFP plasmids were gifts from Pfizer.

HeLa Cell Culture and Transfection: HeLa cells were grown in Dulbecco's modified eagle medium (DMEM, Life Technologies) supplemented with 10% (v:v) heat inactivated fetal bovine serum (Life Technologies), 2 mM I-glutamine, and 100 units/mL penicillin/streptomycin (Life Technologies) in a humidified incubator at 37° C./5% CO₂. Transfection was performed with cells at 70% confluency using Lipofectamine 3000 according to the manufacturer's instructions. HeLa cells were incubated for 5 hours at 37° C./5% CO₂ before being exchanged with antibiotic free growth media for 16-20 hours prior to experimental procedures.

HeLa Cell Labeling: HeLa cells expressing localized HaloTag were grown on poly-1-lysine coated coverslips and labeled with 10 μM 4-((4-(6-methyl-1,2,4,5-tetrazin-3-yl)benzyl)-N-(2-(2-((6-chlorohexyl)oxy)ethoxy)ethyl)amide 21 for 30 minutes at 37° C./5% CO₂. After incubation, cells were washed 3× with DPBS and incubated for an hour in 2 mL of new media to remove excess ligand. After an additional media swap, cells were treated with 1 μM TAMRA-a-TCO 12 for 30 minutes. After fluorophore incubation, unreacted TAMRA-a-TCO 12 was quenched by washing the cells with quenching buffer (100 μM 3-methyl-6-(4-aminomethylphenyl)-s-tetrazine in PBS). The cells were then allowed to sit for 1 hour in media to wash out any remaining dye. To fix cells, media was aspirated, and the wells were washed 3× with PBS before fixation with 4% paraformaldehyde at room temperature for 10 minutes. Cells were washed 3× for 5 minutes in PBS before being mounted onto coverslips with Vectashield HardSet Mounting Medium with DAPI and stored at 4° C. Images were acquired using the Airyscan mode of the Zeiss LSM 880 confocal microscope with the 63 ×1.4NA Plan-Apochromat objective.

TAMRA-TCO Comparative Washout Assay

HeLa cells were seeded at 5×10³ cells per well in a poly-L-lysine (0.1 mg/mL) coated 48-well plate and allowed to grow for 48 hours in Dulbecco's modified eagle medium (DMEM, Life Technologies) supplemented with 5% FBS (Life Technologies), 1 mM L-glutamine, and 1% penicillin/streptomycin (Life Technologies). Cells were then incubated with media containing 5 μM of TAMRA-TCO, TAMRA-oxo-TCO, or TAMRA-a-TCO 12 (1 mM stock solutions in DMSO, diluted twice into media) for 30 mins at 37° C. All wells were washed three times with DPBS before adding fresh media. Cells were imaged after 2 DPBS washes and again after the 3^{rd} DPBS wash, then incubated for additional time intervals: 10 minutes, 40 minutes, and 2 hour. At each time point, the media was removed from the designated wells and replaced with fresh media before imaging. Images were taken with an EVOS M7000 and all images were taken with the same laser power and gain. Fluorescence intensity was calculated for each sample in 3 replicates using ImageJ and the fluorescence data was normalized with the cell count of each image.

### Computational Analysis

Method: M062X/6-311+G(d,p) scrf=(smd,solvent=THF)

Keto-TCO Ground State (Crown Conformation)

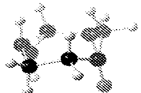

| | |
|---|---|
| Electronic Energy (a.u.) | −639.57964 |
| Sum of electronic and zero-point Energies | −639.36404 |
| Sum of electronic and thermal Energies | −639.3505 |
| Sum of electronic and thermal Enthalpies | −639.34956 |
| Sum of electronic and thermal Free Energies | −639.40429 |

Atomic Coordinates in Angstroms

| Atom | x | y | z |
|---|---|---|---|
| C | −0.658834 | −1.104451 | −0.918879 |
| C | −3.022731 | −0.929489 | 0.112482 |
| C | −0.893470 | 2.166554 | 0.098905 |
| C | −2.498211 | 0.366386 | 0.644257 |
| C | −2.125185 | 1.352717 | −0.171711 |
| C | −1.770144 | −1.817816 | −0.118156 |
| C | 0.263959 | 1.348753 | −0.548393 |
| H | 0.069083 | −1.852243 | −1.254861 |
| H | −3.535667 | −0.771588 | −0.840710 |
| H | −0.714622 | 2.252795 | 1.173742 |
| H | −2.100391 | 0.347195 | 1.659865 |
| H | −2.480108 | 1.345310 | −1.202478 |
| H | −1.073795 | −0.623028 | −1.806098 |
| H | −3.706258 | −1.439993 | 0.794539 |
| H | −0.912767 | 3.167193 | −0.335463 |
| H | −2.060739 | −2.723112 | −0.655746 |
| H | 1.235178 | 1.737318 | −0.236561 |
| H | −1.361026 | −2.133679 | 0.845607 |
| H | 0.187505 | 1.402632 | −1.637072 |
| C | 0.148201 | −0.103063 | −0.113417 |
| O | 0.712648 | −0.481451 | 0.905956 |
| Li | 2.336408 | −0.295635 | 1.778618 |
| H | 3.112378 | −0.191160 | −1.564127 |
| H | 3.397909 | 0.967291 | 0.803940 |
| Al | 3.965454 | −0.214976 | −0.201289 |
| H | 5.553859 | −0.157253 | −0.361618 |
| H | 3.466639 | −1.472017 | 0.739767 |

Transition State of Nucleophilic Addition of Lithium Aluminum Hydride to Axial Face of Keto-TCO

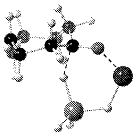

| | | |
|---|---|---|
| Electronic Energy (a.u.) | −639.55363 au | |
| Sum of electronic and zero-point | −639.3388 au | |
| Energies | | |
| Sum of electronic and thermal | −639.326 au | |
| Energies | | |
| Sum of electronic and thermal | −639.32506 au | 15.375 Kcal/mol |
| Enthalpies | | |
| Sum of electronic and thermal Free | −639.37744 au | 16.846 Kcal/mol |
| Energies | | |

| | |
|---|---|
| Imaginary frequency | 370.25722i cm⁻¹ |

Atomic Coordinates in Angstroms

| Atom | x | y | z |
|---|---|---|---|
| C | 0.580167 | −0.432529 | 1.514642 |
| C | 2.474039 | −1.164238 | −0.130275 |
| C | 0.576219 | 1.935789 | −0.972479 |
| C | 1.905536 | −0.135931 | −1.049359 |
| C | 1.801232 | 1.136004 | −0.669996 |
| C | 1.279683 | −1.588819 | 0.763706 |
| C | −0.335886 | 1.794399 | 0.265681 |
| H | 0.119339 | −0.864101 | 2.404999 |
| H | 3.259306 | −0.735372 | 0.499865 |
| H | 0.066599 | 1.534229 | −1.852071 |
| H | 1.258490 | −0.501400 | −1.844337 |
| H | 2.418174 | 1.490808 | 0.156015 |
| H | 1.342408 | 0.274906 | 1.860202 |
| H | 2.877084 | −2.041831 | −0.640700 |
| H | 0.764567 | 2.999631 | −1.138294 |
| H | 1.643363 | −2.290614 | 1.519927 |
| H | −1.324381 | 2.209879 | 0.051574 |
| H | 0.544464 | −2.128521 | 0.160905 |
| H | 0.088009 | 2.395575 | 1.082733 |
| C | −0.570286 | 0.420978 | 0.921033 |
| H | −3.255355 | 0.039070 | −0.842775 |
| O | −1.674525 | 0.291295 | 1.503251 |
| Li | −3.354913 | 0.517573 | 0.892911 |
| Al | −2.110809 | −1.076576 | −1.240230 |
| H | −2.049111 | −1.260735 | −2.831135 |
| H | −0.740070 | −0.392963 | −0.642473 |
| H | −2.375664 | −2.410068 | −0.383914 |

Transition State of Nucleophilic Addition of Lithium Aluminum Hydride to Equatorial Face of Keto-TCO

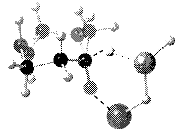

| | | |
|---|---|---|
| Electronic Energy (a.u.) | −639.55967 au | |
| Sum of electronic and zero-point | −639.34398 au | |
| Energies | | |
| Sum of electronic and thermal | −639.33139 au | |
| Energies | | |
| Sum of electronic and thermal | −639.33045 au | 11.994 Kcal/mol |
| Enthalpies | | |
| Sum of electronic and thermal Free | −639.38184 au | 14.086 Kcal/mol |
| Energies | | |

| | |
|---|---|
| Imaginary frequency | 477.2191i cm⁻¹ |

### Example 30: Synthesis of (E)-8-methylcyclooct-4-en-1-one

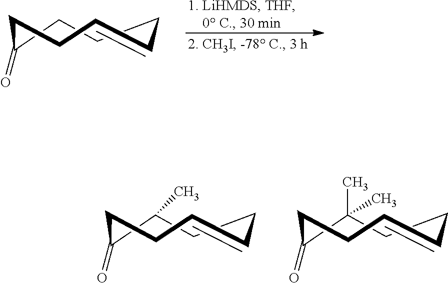

A round bottom flask equipped with a magnetic stir bar was charged with LiHMDS (0.36 mL, 1M in THF) then was chilled to 0° C. (E)-Cyclooct-4-enone (30 mg, 0.24 mmol) in 0.16 mL of THF was added dropwise and stirred for 30 minutes before chilling to −78° C. Methyl iodide (21 μL, 0.34 mmol) was added dropwise and stirred at −78° C. for 40 mins before warming to 0° C. and stirring for another 2.5 hour. The reaction was quenched with 0.5 mL of NH₄Cl sat. solution and the aqueous layer was extracted with Et₂O. The organic layer was washed with brine, dried with MgSO₄, filtered and concentrated. The crude was purified by column chromatography (0-2% diethyl ether in hexanes) to afford 14.5 mg of a 3:1 mixture of monoalkylation (33% yield) to dialkylation product as determined by ¹H NMR analysis as clear, colorless oil. Further purification afforded a 10:1 mixture of monoalkylation to dialkylation that was utilized to further characterize the monoalkylation product.

Peaks attributed to monoalkylation product: ¹H NMR (600 MHz, C₆D₆) δ 5.42 (ddd, J=15.4, 11.2, 3.7 Hz, 1H), 5.24 (ddd, J=15.7, 11.3, 3.7 Hz, 1H), 2.52-2.42 (m, 1H), 2.27 (ddd, J=12.6, 10.4, 5.9 Hz, 1H), 2.18 (app dd, J=10.4, 5.9 Hz, 1H), 2.13-2.07 (m, 1H), 2.05-2.00 (m, 1H), 1.94-1.78 (m, 2H), 1.67 (app qd, J=11.5, 5.2 Hz, 1H), 1.24 (app dd, J=12.6, 5.3 Hz, 1H), 0.77 (d, J=6.5 Hz, 3H). ¹³C NMR (151 MHz, C₆D₆) δ 217.2 (C), 134.5 (CH), 131.2 (CH), 48.4 (CH), 48.0 (CH₂), 37.1 (CH₂), 34.7 (CH₂), 33.3 (CH₂), 17.5 (CH₃). FTMS (ESI+) calculated [M+H]⁺ for C₉H₁₅O 139.1123; found 139.1116.

Peaks attributed to dialkylation product: ¹H NMR (600 MHz, C₆D₆) δ 5.57 (ddd, J=16.3, 11.0, 3.9 Hz, 1H), 5.30-5.20 (m, 1H), 2.65 (ddd, J=12.9, 10.3, 4.8 Hz, 1H), 2.61-2.53 (m, 1H), 2.07-1.93 (m, 2H), 1.93-1.79 (m, 2H), 1.14-1.07 (m, 2H), 0.93 (s, 3H), 0.86 (s, 3H). ¹³C NMR (151 MHz, C₆D₆) δ 217.2 (C), 134.3 (CH), 130.1 (CH), 46.2 (C), 44.6 (CH₂), 42.9 (CH₂), 35.5 (CH₂), 30.2 (CH₂), 29.6 (CH₃), 22.2 (CH₃).

### Example 31: Other Derivatives Synthesized by the Method Described in Example 30 Include the Following

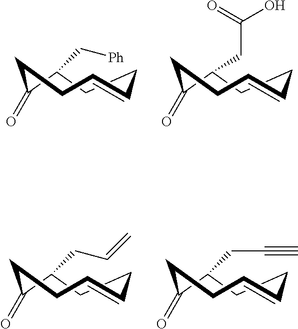

Although the invention is illustrated and described herein with reference to specific embodiments, the invention is not intended to be limited to the details shown. Rather, various modifications may be made in the details within the scope and range of equivalents of the claims and without departing from the invention.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

1. A trans-cyclooct-4-eneone having the following formula (2): 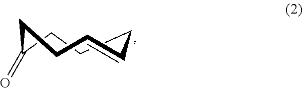
wherein the trans-cyclooct-4-eneone 2 characterized by ¹H NMR (400 MHz, CDCl₃) includes peaks at 5.27 ppm and 2.91 ppm.

2. The trans-cyclooct-4-enone of claim 1, wherein the trans-cyclooct-4-enone is in an isolated form.

3. The trans-cyclooct-4-enone of claim 1, wherein the trans-cyclooct-4-enone is at least 90% pure.

4. The trans-cyclooct-4-enone of claim 1, produced by a photochemical flow method comprising irradiating cis-cyclooct-4-enone with light from a low-pressure mercury lamp for a time sufficient to form the trans-cyclooct-4-enone.

5. A substituted axial hydroxy-trans-cyclooctene, having the following formula (2a): 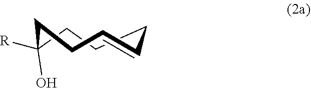
where R is selected from hydrogen, alkyl, aryl, and heteroaryl.

6. The substituted axial hydroxy-trans-cyclooctene of claim 5, wherein R is selected from hydrogen, allyl, acetate, cyano, acetohydrazide, hydroxyethyl, (prop-2-yn-1-yloxy)ethyl, amino ethyl, hydroxysuccinyl acetate, phenyl, and phenylethynyl.

7. The substituted axial hydroxy-trans-cyclooctene of claim 5, wherein the trans-cyclooctene exists as a single diastereoisomer.

8. The substituted axial hydroxy-trans-cyclooctene of claim 5 having one of the following structures:

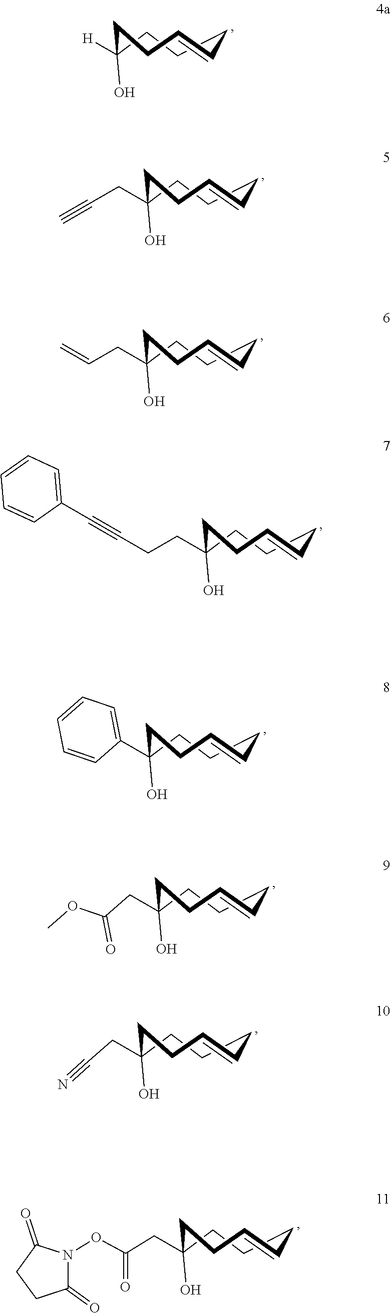

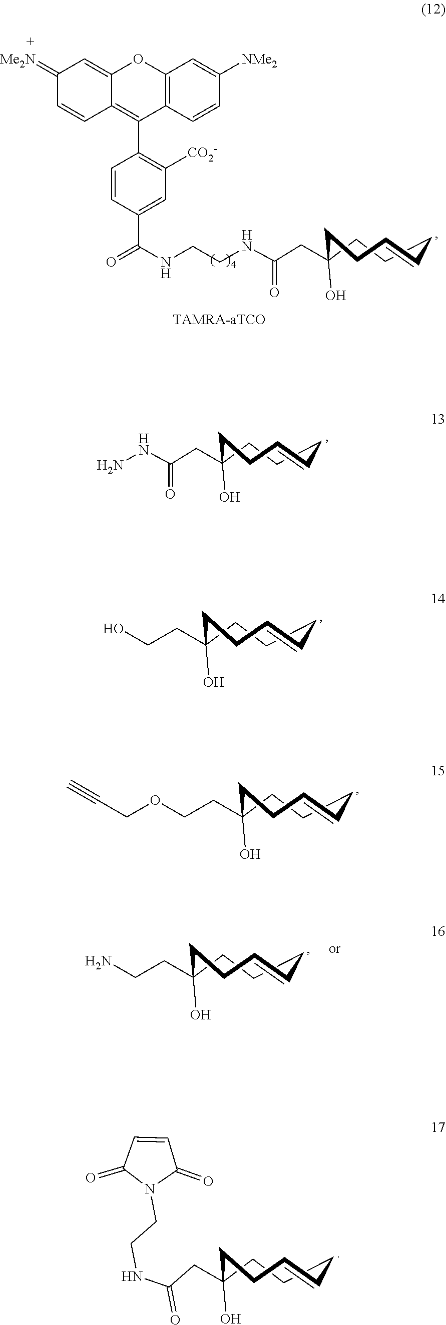

9. An alpha-substituted trans-cyclooct-4-enone, having the formula: 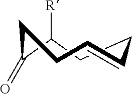
where R′ is selected from the group consisting of alkyl, aryl, carboxylic acid, alkene, and alkyne.

10. An oxime conjugate having the following formula: 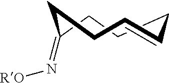
where R″ is selected from the group consisting of hydrogen, aryl, and alkyl.

11. The oxime conjugate of claim 10 having one of the following structures:

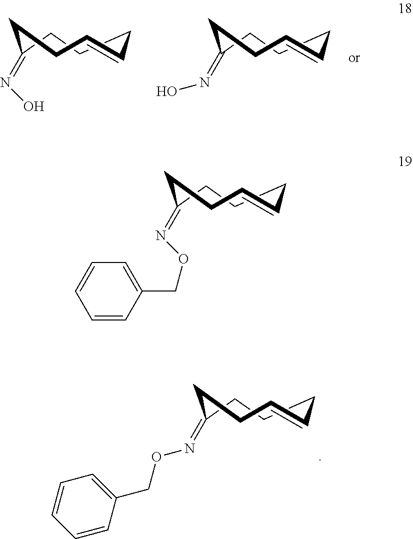

12. A method of producing the substituted axial hydroxy-trans-cyclooctene of claim 5, the method comprising contacting trans-cyclooct-4-enone with a nucleophile for a stereocontrolled 1,2-addition of the nucleophile to the trans-cyclooct-4-enone,
wherein nucleophilic addition to the trans-cyclooct-4-eneone take place exclusively from the equatorial-face of the trans-cyclooctennone to produce an axial hydroxy-trans-cyclooctene as a single diastereomer,
wherein the nucleophile is a Grignard reagent, an organolithium, or an organozinc.

13. The method of claim 12, wherein nucleophile is selected from lithium phenyl acetylene, methyl α-lithioacetate, lithioacetonitrile, and lithium bis(trimethylsilyl)amide.

14. The method of claim 12, wherein the substituted axial hydroxy-trans-cyclooctene is produced as a single diastereoisomer.

15. The method of claim 12, wherein the substituted axial hydroxy-trans-cyclooctene is produced with a yield of at least 80%.

16. The method of claim 12, wherein the substituted axial hydroxy-trans-cyclooctene is at least 95% pure.

17. A method of producing the alpha-substituted trans-cyclooct-4-enone of claim 9 comprising treating a trans-cyclooct-4-enone, having the following formula (2), with a base followed by the addition of an electrophile, 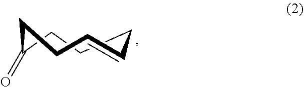
wherein the trans-cyclooct-4-eneone 2 characterized by ¹H NMR (400 MHz, CDCl₃) includes peaks at 5.27 ppm and 2.91 ppm.

18. The method of claim 17, where the electrophile is selected from alkyl halides, alkyl sulfonates, epoxides, aldehydes, or ketones.

19. The method of claim 17, wherein the substituted axial hydroxy-trans-cyclooctene is produced as a single diastereoisomer.

20. The method of claim 19, wherein the substituted axial hydroxy-trans-cyclooctene is produced with a yield of at least 80%.
